SEQUENCE LISTING (1 sequence)

SEQ ID NO: 1 - DNA, 17 bp, Unknown
  examplary nucleotide sequence in a biological organism that binds to a trinucleotide primer
agaagaagaagattttt

(19) United States
(12) Patent Application Publication
(10) Pub. No.: US 2012/0077196 A9
(43) Pub. Date: Mar. 29, 2012

(54) Universal method for selective amplification of mRNAs

(76) Inventors: Guido Krupp, Gnutz (DE); Peter Scheinert, Hamburg (DE)

(21) Appl. No.: 11/513,118

(22) Filed: Aug. 31, 2006

Related U.S. Application Data
(63) Continuation-in-part of application No. 10/488,231, filed on Aug. 5, 2004, now abandoned.
(60) Provisional application No. 60/712,820, filed on Sep. 1, 2005.

(30) Foreign Application Priority Data
Sep. 3, 2001 (DE) 10143106.6

(51) Int. Cl. C12Q 1/68; C12P 19/34

(52) U.S. Cl. 435/6.12; 435/91.2; 435/6.15

(57) ABSTRACT

The invention relates generally to methods for the amplification of ribonucleic acids, including for example messenger ribonucleic acids (mRNAs). In an embodiment, the invention also relates to kits for amplifying ribonucleic acids, including for example mRNAs. In another embodiment, the invention relates to kits comprising the components for performing the methods of the present invention.

### CROSS-REFERENCE TO RELATED APPLICATION

The present application claims the benefit of U.S. Provisional Application No. 60/712,820, filed Sep. 1, 2005, which application is herein incorporated by reference in its entirety.

### BACKGROUND OF THE INVENTION

To date, a multitude of methods resulting in the amplification of nucleic acids are known. The best known example is the polymerase chain reaction (PCR), developed by Kary Mullis in the mid-1980s (see Saiki et al., Science, Vol. 230 (1985), 1350-1354; and EP 201 184).

During the PCR reaction, single-stranded primers (oligonucleotides with a chain-length of usually 12 to 24 nucleotides) bind to a complementary, single-stranded DNA sequence. These primers are subsequently elongated to double-stranded DNA, in the presence of a DNA polymerase and deoxyribonucleoside triphosphates (dNTPs, namely dATP, dCTP, dGTP and dTTP). The double-stranded DNA is separated by heating into single strands. The temperature is reduced sufficiently to allow a new step of primer binding. Again, primer elongation results in double-stranded DNA.

Repetition of the steps described above enables exponential amplification of the input DNA. This is achieved by adjusting the reaction conditions such that almost each molecule of single-stranded DNA within each round of amplification will be transformed into double-stranded DNA, melted into single-stranded DNAs which will be used again as template for the next round of amplification.

It is possible to conduct a reverse transcription reaction prior to the above mentioned PCR reaction. This means, in the presence of an RNA-dependent DNA polymerase, messenger ribonucleic acid (mRNA) is transformed into single-stranded DNA (complementary DNA or cDNA), which can then be used in a PCR reaction, hence resulting in the amplification of RNA sequences (see, e.g., EP 201 184).

This basic reaction model of a PCR reaction has been altered in the last years and a multitude of alternatives have been developed, depending on the starting materials (RNA, DNA, single- or double-stranded) and also relating to different reaction products (amplification of specific RNA or DNA sequences from the mixture of different nucleic acids within one sample, or the amplification of all RNA/DNA sequences present in one sample).

Over the last years, so-called microarrays for the analysis of nucleic acids are used with increasing frequency. The essential component of such a microarray is an inert carrier onto which a multitude of different nucleic acid sequences (DNA is used most frequently) are bound in different regions of the carrier. Usually, within one particular very small region, only DNA with one specific sequence is bound, resulting in microarrays with several thousand different regions capable of binding several thousand different sequences.

These microarray plates can be incubated with a multitude of nucleic acid sequences (in general labeled DNA or RNA) obtained from a sample of interest, resulting, under suitable conditions (ion content, temperature and so forth), in complementary hybrid molecules of nucleic acid sequences between those sequences originating from the sample of interest and those sequences bound to the microarray plate. Unbound, non-complementary sequences can be washed off. Due to the presence of the label, the regions on the microarray containing double-stranded DNA can be detected and thus, the sequences as well as the amount of nucleic acids bound from the original sample can be analyzed.

Microarrays are used to analyze expression profiles of cells, hence allowing the analysis of all mRNA sequences expressed in certain cells (see Lockhart et al., Nat. Biotechnology 14 (1996), 1675-1680).

The amount of RNA (and thus mRNA) available for this sort of analysis is usually limited. Therefore special methods have been developed to amplify the RNAs, which are to be analyzed using microarrays. As a first step, the ribonucleic acids are usually converted to more stable cDNAs using reverse transcription.

Methods yielding large amounts of amplified RNA populations of single cells are described in, e.g., U.S. Pat. No. 5,514,545. This method uses a primer containing an oligo-dT-sequence and a T7-promoter region. The oligo-dT-sequence binds to the 3′-poly-A-sequence of the mRNA initiating the reverse transcription of the mRNA. Alkaline conditions result in the denaturation of the RNA/DNA heteroduplex, and the hairpin structure at the 3′-end of the cDNA can be used as primer to initiate synthesis of the second DNA strand. The resulting construct is converted to a linear double-stranded DNA by using nuclease S1 to open the hairpin structure. Then the linear double-stranded DNA is used as template for T7 RNA polymerase. The resulting RNA can be used again as template for the synthesis of cDNA. For this reaction, oligonucleotide hexamers of random sequences (random primers) are used. Following heat-induced denaturation, the second DNA strand is produced using the above mentioned T7-oligo-dT-primer and the resulting DNA can be used again as template for T7 RNA polymerase.

An alternative strategy is presented in U.S. Pat. No. 5,545,522. Here, it is demonstrated that a single oligonucleotide primer can be used to yield high amplifications. RNA is reverse transcribed to cDNA, and the primer has the following characteristics: a) 5′-dN₂₀, meaning a random sequence of 20 nucleotides; b) a minimal T7-promoter; c) GGGCG as transcription-initiation sequence; and d) oligo-dT₁₅. Synthesis of the second DNA strand is achieved by partial RNA digestion with RNase H. The remaining RNA-oligonucleotides are used as primers for DNA polymerase I. The ends of the resulting DNA are blunted by T4-DNA polymerase.

A similar procedure is disclosed in U.S. Pat. No. 5,932,451. In this procedure, two so-called box-primers are added within the 5′ proximal area, enabling the double immobilization by using biotin-box-primers.

However, the above mentioned methods to amplify ribonucleic acids may have various disadvantages. For example, the above mentioned methods result in RNA populations which are different from the RNA populations present in the original starting material. This is due to the use of the T7-promoter-oligo-dT-primers, which primarily amplify RNA sequences of the 3′-section of the mRNA. Furthermore, it has been shown that long primers (more than 60 nucleotides) containing 3′-terminal homo-oligomeric sequences (i.e., oligo-dT) are prone to build primer-primer-hybrids and also allow for non-specific amplification of the primers, even yielding very long amplified nucleic acids with a length of several kilobases (Baugh et al., Nucleic Acids Res. 29 (2001) E29). Therefore, known procedures may result in the production of a multitude of artifacts, interfering with the further analysis of the nucleic acids.

To overcome these artifacts, WO03/020873 discloses a method for the amplification of ribonucleic acids, wherein a single-stranded DNA is obtained via reverse transcription from RNA, using, e.g., oligo-dT as primer that is specific for eukaryotic mRNA (due to the universal 3′-poly-A sequence). Then the RNA is eliminated, and a double-stranded DNA is generated using a special primer construct comprising the sequence of a promoter, the two DNA strands are separated into single strands and a further double-stranded DNA is generated using a primer also containing the sequence of a promoter and, e.g., for mRNA amplification a 3′-terminal oligo-dT sequence. RNA polymerase is then used to generate a plurality of single-stranded RNAs.

The above methods can be used to amplify specifically eukaryotic mRNAs having a universal poly-A tail. However, two situations exist where no sequence which is generally applicable is available for specific amplification of mRNAs or mRNA-derived sequences: (i) Prokaryotic species, i.e., Bacteria or Archaea have mRNAs without any universal 3 ′-terminal sequence; (ii) Eukaryotic RNA samples that have suffered degradation due to their pre-treatment procedures prior to the isolation of RNA. These potentially problematic procedures include elevated temperatures without complete inactivation of nucleases, staining steps that can cause chemical or enzymatic RNA degradation, and the preparation and long-term storage of archival samples, such as formalin-fixed paraffin-embedded tissues. In the last example type, in addition to severe degradation, mRNA amplification is further complicated by limited sequence accessibility, due to formalin-caused cross-linking of RNAs to proteins and to nucleic acids.

In the vast majority of analyses even for those samples described in the preceding sections (i) and (ii), it is the aim of the scientists to analyze to the greatest extent possible a complete population of mRNA sequences. For this purpose, it would be advantageous to amplify, selectively and universally, all mRNA sequences (in intact or degraded mRNAs).

To achieve selective amplification of prokaryotic mRNAs, other RNA species, such as ribosomal RNA (rRNA), may be reduced or eliminated prior to mRNA amplification (Ambion RNA Removal Kits). This purification step may be followed by reverse transcription using random primers, thus amplifying all RNA sequences still present. This way of proceeding may have the disadvantage that random primers are elongated non-selectively at all exposed RNA stretches, without any preference for 3′-proximal priming and thus no preference for full-length cDNAs is obtained. As is directly evident, this method may further increase handling time and costs.

The mRNA sequences in degraded RNA samples may be processed in two ways. Specific mRNA amplification may be maintained by using oligo-dT primers, and mRNA sequences in fragments without the 3′-terminal poly-A are lost (Paradise kit from Arcturus). Alternatively, RNA sequences generally, including rRNA sequences, may be amplified (kits for degraded eukaryotic RNAs, available from Ambion and from Nugen).

One problem underlying the present invention therefore resides in providing a method to amplify ribonucleic acids, which allows selective amplification of messenger ribonucleic acids (mRNAs) which can also be applied to intact prokaryotic mRNAs or degraded eukaryotic mRNAs. This problem is addressed by the present invention, for example, in various methods and kits for the amplification of mRNAs.

### BRIEF SUMMARY OF THE INVENTION

The present invention includes and provides a method for the amplification of messenger ribonucleic acids (mRNAs), comprising:
(a) producing a first single-stranded DNA from a starting material comprising mRNA, using an RNA-dependent DNA polymerase, deoxyribonucleoside triphosphates, and a mixture of first single-stranded primers comprising the sequence 5′—a Box 1 sequence—1 to 6 random nucleotides—a specific trinucleotide sequence—3′;
(b) removing RNAs from the admixture of step (a);
(c) producing a first double-stranded DNA from said first single-stranded DNA using a DNA-dependent DNA polymerase, deoxyribonucleoside triphosphates, and a mixture of second single-stranded primers comprising the sequence 5′—a Box 2 sequence—1 to 6 random nucleotides—a specific trinucleotide sequence—3′, wherein said mixture of said second single-stranded primers differs from said mixture of said first single-stranded primers used in step (a);
(d) separating said first double-stranded DNA into second single-stranded DNAs;
(e) producing a second double-stranded DNA from one of said second single-stranded DNAs obtained in step (d), using a DNA-dependent DNA polymerase, deoxyribonucleoside triphosphates, and a third single-stranded primer comprising the sequence 5′—a promoter sequence—said Box 1 sequence—3′ or the sequence 5′—a promoter sequence—said Box 2 sequence—3′; and
(f) producing a plurality of first single-stranded RNAs, both ends of which comprise defined sequences of said Box 1 sequence or said Box 2 sequence, using an RNA polymerase and ribonucleoside triphosphates.

The present invention also includes and provides a method for nucleic acid analysis, comprising the following steps:
(a) obtaining ribonucleic acids;
(b) amplifying said ribonucleic acids using the method cording to claim 1 or claim 19; and
(c) analyzing said amplification product obtained in step (b) using microarrays.

The present invention also includes and provides a method for nucleic acid analysis, comprising:
(a) obtaining ribonucleic acids;
(b) amplifying said ribonucleic acids using the method according to claim 1 or claim 19;
(c) converting said amplification product obtained in step (b) to cDNA; and
(d) analyzing said cDNAs using microarrays.

The present invention also includes and provides a kit comprising:
(a) a mixture of single-stranded primers comprising the following sequences 5′—Box 1 sequence—1 to 6 random nucleotides—a specific trinucleotide sequence—3′;
(b) a mixture of single-stranded primers comprising the following sequences 5′—Box 2 sequence—1 to 6 random nucleotides—a specific trinucleotide sequence—3′;
(c) a single-stranded primer comprising the following sequences 5′—promoter sequence—Box 1 sequence—3′;
(d) an RNA-dependent DNA polymerase;
(e) deoxyribonucleoside triphosphates;
(f) a DNA-dependent DNA polymerase;
(g) an RNA polymerase; and
(h) ribonucleoside triphosphates.

### BRIEF DESCRIPTION OF THE FIGURES

FIG. 1 shows an exemplary function of the Trinucleotide Primers in methods of the present invention.

FIG. 2 shows an exemplary amplification of sequences encoding a cytokine mRNA as a model in comparison to a nucleic acids ladder as a size marker.

FIG. 3 shows an example of amplified RNA using a random primer in comparison to the size marker in the upper part and the amplified RNA using a Trinucleotide Primer of the present invention in comparison to the size marker in the lower part.

FIG. 4 shows an example of electropherograms of amplified bacterial mRNAs obtained by methods of the present invention.

FIG. 5 shows an example of hybridization results of specific sequences using the Affymetrix HG-U133A chip after amplification.

### DETAILED DESCRIPTION OF THE INVENTION

The invention relates generally to methods for the amplification of messenger ribonucleic acids (mRNAs). In an embodiment, the invention also relates to kits for amplifying ribonucleic acids, including, for example, mRNAs. In another embodiment, the invention relates to kits comprising the components for performing the methods of the present invention.

Various non-limiting embodiments include:
Embodiment 1. Method for the amplification of messenger ribonucleic acids comprising the following steps:
   (a) a single stranded DNA is produced from a starting material comprising mRNA by means of reverse transcription, using a mixture of single-stranded primers comprising the following sequences 5′—Box 1 sequence—1 to 6 random nucleotides—a specific trinucleotide sequence—3′, an RNA-dependent DNA polymerase and deoxyribonucleoside triphosphates;
   (b) the RNA is removed from the sample;
   (c) a DNA duplex is produced using a mixture of single-stranded primers comprising the following sequences 5′—Box 2 sequence—1 to 6 random nucleotides—a specific trinucleotide sequence—3′, wherein the mixture of primers differs from the mixture of primers used in step (a), a DNA polymerase and deoxyribonucleoside triphosphates;
   (d) the duplex is separated into single-stranded DNAs;
   (e) DNA duplexes are produced from one of the single-stranded DNAs obtained in step (d) using a single-stranded primer comprising the sequences 5′—promoter sequence—Box 1 sequence—3′ or the sequences 5′—promoter sequence—Box 2 sequence—3′, a DNA polymerase and deoxyribonucleoside triphosphates;
   (f) a plurality of single stranded RNAs is produced, both ends of which comprise defined sequences, by means of an RNA polymerase and ribonucleoside triphosphates.
Embodiment 2. Method according to embodiment 1, wherein the single-stranded RNA obtained has the inverse sense orientation (antisense sequence) in relation to the RNA starting material.
Embodiment 3. Method according to embodiments 1 and 2, characterized in that the Box 1 sequence is the same or a different sequence than the Box 2 sequence.
Embodiment 4. Method according to any of the embodiments above, characterized in that the method yields as a product a mixture of ribonucleic acids, which contain more than 70%, preferably more than 80% or more than 90% mRNA.
Embodiment 5. Method according to any of the embodiments above, characterized in that in step (b) the RNA is hydrolyzed by means of RNase.
Embodiment 6. Method according to any of the embodiments above, characterized in that in step (b) the RNA is removed by means of RNase I and/or RNase H.
Embodiment 7. Method according to any of the embodiments above, characterized in that the Box sequence contains at least 6 nucleotides, having a low homology to known gene sequences, that are expressed in multi-cellular organisms.
Embodiment 8. Method according to any of the embodiments above, characterized in that the method is used for the amplification of bacterial mRNA, for the amplification of eukaryotic mRNA or for the amplification of degraded mRNA.
Embodiment 9. Method according to any of the embodiments above, characterized in that a reverse transcriptase is used as DNA polymerase.
Embodiment 10. Method according to any of the embodiments above, characterized in that dATP, dCTP, dGTP and dTTP are used as deoxyribonucleoside-monomers.
Embodiment 11. Method according to any of the embodiments above, characterized in that in step (d) DNA double strands are separated in single strands by means of heat.
Embodiment 12. Method according to any of the embodiments above, characterized in that in step (e) a single-stranded primer is used, which comprises the sequence of either the T7, T3 or SP6 RNA polymerase.
Embodiment 13. Method according to any of the embodiments above, characterized in that ATP, CTP, GTP and UTP are used as ribonucleoside-monomers.
Embodiment 14. Method according to any of the embodiments above, characterized in that the amplification factor of the starting RNA sequence is at least 500, preferably more than 1000.
Embodiment 15. Method according to any of the embodiments above, characterized in that the method comprises after step (f) the following steps for further amplification of ribonucleic acids:
   (g) using the single-stranded RNAs generated in step (f) as template, single-stranded DNA is synthesized using reverse transcriptase, a single-stranded primer, comprising the Box 2 sequence, an RNA-dependant DNA polymerase and deoxyribonucleoside triphosphates;
   (h) the RNA is removed;
   (i) using the single-stranded DNA generated in (h) as template, double-stranded DNA is synthesized using a single-stranded primer, comprising a 5′-Promoter—Box 1 sequence—3′, a DNA polymerase and deoxyribonucleoside triphosphates;
   (j) a multitude of single-stranded RNAs is synthesized using a RNA polymerase and ribonucleoside triphosphates.
Embodiment 16. Method according to any of the embodiments above, characterized in that in step (h) the RNA is hydrolyzed by means of RNase.
Embodiment 17. Method according to embodiments above, characterized in that all single-stranded RNAs produced in step (j) have inverse orientation.
Embodiment 18. Kit for ribonucleic acid amplification according to embodiments 1 to 17, comprising the following components:
   (a) a mixture of single-stranded primers comprising the following sequences 5′—Box 1 sequence—1 to 6 random nucleotides—a specific trinucleotide sequence—3′;
   (b) a mixture of single-stranded primers comprising the following sequences 5′—Box 2 sequence—1 to 6 random nucleotides—a specific trinucleotide sequence—3′;
   (c) a single-stranded primer comprising the following sequences 5′—promoter sequence—Box 1 sequence—3′;
   (d) an RNA-dependent DNA polymerase;
   (e) deoxyribonucleoside triphosphates;
   (f) a DNA-dependent DNA polymerase;
   (g) an RNA polymerase; and
   (h) ribonucleoside triphosphates.
Embodiment 19. Kit according to embodiment 18, characterized in that the kit comprises three different single-stranded primers.
Embodiment 20. Kit according to embodiment 18, characterized in that the kit further comprises a single-stranded primer, comprising the Box 2 sequence, an RNA-dependant DNA polymerase and deoxyribonucleoside triphosphates and a single-stranded primer, comprising a 5′—Promoter—Box 1 sequence—3′.
Embodiment 21. Kit according to embodiments 18 to 20, characterized in that in addition, the kit comprises RNase I and/or RNase H.
Embodiment 22. Kit according to embodiment 18 to 21, characterized in that the kit comprises a single-stranded primer with a T7, T3 or SP6 RNA polymerase promoter sequence.
Embodiment 23. Kit according to embodiments 18 to 22, characterized in that it comprises a reverse transcriptase as DNA polymerase.
Embodiment 24. Kit according to embodiments 18 to 23, characterized in that it comprises the T7 RNA polymerase.
Embodiment 25. Kit according to embodiments 18 to 24, characterized in that it comprises a composition for labeling of amplified RNA with a detectable moiety.
Embodiment 26. Kit according to embodiments 18 to 25, characterized in that the kit includes a DNA-microarray.
Embodiment 27. Method for nucleic acid analysis that involves production of ribonucleic acids, amplification with a method according to embodiments 1 to 17, and analysis by means of microarrays.
Embodiment 28. Method according to embodiment 27 characterized in that the ribonucleic acids is isolated from a biological sample.
Embodiment 29. Method according to embodiments 27 or 28, characterized in that ribonucleic acids are amplified, converted to cDNA by means of reverse transcription, and the cDNAs are analyzed by means of microarrays.
Embodiment 30. Method according to embodiments 27 to 29, characterized in that the amount and/or sequence of the mRNA in the starting material are analyzed.
Embodiment 31. A method for the amplification of messenger ribonucleic acids comprising the following steps:
   (a) producing a single stranded DNA from a starting material comprising mRNA by reverse transcription, using an RNA-dependent DNA polymerase, deoxyribonucleoside triphosphates, and a mixture of single-stranded primers each of which comprises the following sequences operably linked in 5′ to 3′ orientation: a Box 1 sequence linked to 1 to 6 random nucleotides linked to a specific trinucleotide sequence;
   (b) removing the RNA from the sample;
   (c) producing a DNA duplex using a DNA polymerase, deoxyribonucleoside triphosphates, and a second mixture of single-stranded primers each of which comprises the following sequences operably linked in 5′ to 3′ orientation: a Box 2 sequence linked to 1 to 6 random nucleotides linked to a specific trinucleotide sequence, wherein the second mixture of single-stranded primers differs from the mixture of primers used in step (a);
   (d) separating the duplex into single-stranded DNAs;
   (e) producing DNA duplexes from one of the single-stranded DNAs obtained in step (d) using a DNA polymerase, deoxyribonucleoside triphosphates, and a single-stranded primer comprising the following sequences operably linked in 5′ to 3′ orientation: a promoter sequence linked to a Box 1 sequence, or comprising the following sequences operably linked in 5′ to 3′ orientation: a promoter sequence linked to a Box 2 sequence; and
   (f) producing a plurality of single stranded RNAs, both ends of which comprise defined sequences, using an RNA polymerase and ribonucleoside triphosphates.
Embodiment 32. The method according to embodiment 31, characterized in that the method comprises after step (f) the following steps for further amplification of ribonucleic acids:
   (g) using the single-stranded RNAs generated in step (f) as template, single-stranded DNA is synthesized by reverse transcription, using an RNA-dependant DNA polymerase, deoxyribonucleoside triphosphates, and a single-stranded primer comprising the Box 2 sequence;
   (h) the RNA is removed;
   (i) using the single-stranded DNA generated in (h) as template, double-stranded DNA is synthesized using a DNA polymerase, deoxyribonucleoside triphosphates, and a single-stranded primer comprising the following sequences operably linked in 5′ to 3′ orientation: a promoter linked to the Box 1 sequence;
   (j) a multitude of single-stranded RNAs is synthesized using an RNA polymerase and ribonucleoside triphosphates.
Embodiment 33. A kit for ribonucleic acid amplification according to embodiments 31 or 32, comprising the following components:
   (a) a mixture of single-stranded primers each of which comprises the following sequences operably linked in 5′ to 3′ orientation: a Box 1 sequence linked to 1 to 6 random nucleotides linked to a specific trinucleotide sequence;
   (b) a mixture of single-stranded primers each of which comprises the following sequences operably linked in 5′ to 3′ orientation: a Box 2 sequence linked to 1 to 6 random nucleotides linked to a specific trinucleotide sequence;
   (c) a single-stranded primer comprising the following sequences operably linked in 5′ to 3′ orientation: a promoter sequence linked to a Box 1 sequence;
   (d) an RNA-dependent DNA polymerase;
   (e) deoxyribonucleoside triphosphates;
   (f) a DNA-dependent DNA polymerase;
   (g) an RNA polymerase; and
   (h) ribonucleoside triphosphates.

In an embodiment, the invention relates to methods for the amplification of mRNAs, comprising:
(a) producing a first single-stranded DNA from a starting material comprising mRNA, using an RNA-dependent DNA polymerase, deoxyribonucleoside triphosphates, and a mixture of first single-stranded primers comprising the sequence 5′—a Box 1 sequence—1 to 6 random nucleotides—a specific trinucleotide sequence—3′;
(b) removing RNAs from the admixture of step (a);
(c) producing a first double-stranded DNA from said first single-stranded DNA using a DNA-dependent DNA polymerase, deoxyribonucleoside triphosphates, and a mixture of second single-stranded primers comprising the sequence 5′—a Box 2 sequence—1 to 6 random nucleotides—a specific trinucleotide sequence—3′, wherein said mixture of said second single-stranded primers differs from said mixture of said first single-stranded primers used in step (a);
(d) separating said first double-stranded DNA into second single-stranded DNAs;
(e) producing a second double-stranded DNA from one of said second single-stranded DNAs obtained in step (d), using a DNA-dependent DNA polymerase, deoxyribonucleoside triphosphates, and a third single-stranded primer comprising the sequence 5′—a promoter sequence—said Box 1 sequence—3′ or the sequence 5′—a promoter sequence—said Box 2 sequence—3′; and
(f) producing a plurality of first single-stranded RNAs, both ends of which comprise defined sequences of said Box 1 sequence or said Box 2 sequence, using an RNA polymerase and ribonucleoside triphosphates.

The present inventors have surprisingly found that this method and specifically the use of primers having the specific trinucleotide sequence (which primers are also designated Trinucleotide primers for this reason) is not only a method for complete amplification of all mRNA sequences in the starting material—regardless of the nature of the starting material—but further results in a selective amplification of mRNAs. In the context of the present invention, a method for the selective amplification of mRNAs is a method which amplifies primarily mRNAs from a starting material, which typically contains a mixture of different ribonucleic acids and in most cases contains a substantial amount of rRNA (e.g., more than 90%). The starting material may for example be a complex biological starting material, such as a prokaryotic or eukaryotic cell extract or any purified or partially purified fraction thereof. A conventional cell extract will contain ribosomal RNA usually in an amount of about 90% or even more. Most nucleic acid analyses are, however, directed at determining the expression pattern of certain genes and thus aim to analyze mRNA. The methods of the present invention may improve these subsequent analysis steps, for example, by a selective amplification of the mRNAs from the starting material.

In the context of the present application, the term “amplification of mRNA” is used to refer to methods which yield as a product a mixture of ribonucleic acids, which contain more than 70%, preferably more than 80%, or more than 90% mRNA. Methods yielding more than 95% or more than 97% mRNA are most preferred. Quantitative or semi-quantitative determination of the mRNA content in a mixture of different ribonucleic acids can be carried out using DNA Arrays which allow determination and quantification of the presence and amount of mRNA and rRNA. Amplification products that have a high yield of mRNA, for example, such as may be obtained in accordance with methods of the present invention, may be improved with respect to subsequent analysis steps.

In various embodiments, the present invention can be carried out using any starting material. In an embodiment, the method may show specific advantages when employed for amplification of RNAs, for example, from bacterial RNA, partially or severely degraded RNA, or RNA from formalin-fixed or paraffin-embedded samples. As one of ordinary skill in the art can appreciate, the method of the present invention can also be employed for amplification of synthetic RNAs, including, for example, heterologous or degraded synthetic RNAs.

Within the scope of the present invention, an RNA or DNA sequence is called a Box sequence if it comprises a defined sequence of 10 to 25 nucleotides, having only low homology to gene sequences of the organisms from which the starting RNA template for amplification was isolated.

Low homology between a potential Box sequence and corresponding gene sequences can be determined experimentally using standard Northern Blot analysis. To this end, RNA samples from an organism of interest (e.g., plants, humans or animals), meaning the organism from which RNA was isolated for further amplification, is separated using electrophoresis and transferred onto a membrane and hybridized with a labeled oligonucleotide containing a Box sequence. In an embodiment, low homology is characterized by the absence of a hybridization signal under stringent hybridization conditions. For example, stringent conditions can be achieved by washing the membrane, after the hybridization, for 40 minutes at 25° C. with a buffer containing 0.1×SSC and 0.1% SDS. Other stringent hybridization conditions are well known to the skilled artisan. See, e.g., J. Sambrook et al., Molecular Cloning: A Laboratory Manual, Third Edition, Cold Spring Harbor Laboratory Press, 2001.

As an alternative to the above mentioned experimental procedure to verify a Box sequence, it is possible to determine a sequence with low homology by searching databases containing known gene sequences, that are expressed in multi-cellular organisms. To date, known gene sequences that are expressed in multi-cellular organisms are generally stored in databases with open access to the public. These sequences are either stored as gene sequences with known function, or, if the function is not known, these sequences are stored as so called “expressed sequence tags” or ESTs.

In an embodiment, a sequence with only low homology to known sequences is suitable as a Box sequence, if this sequence in comparison to all sequences listed in a database shows over a total length of 10 to 25 nucleotides at least 20%, but preferably 30 or 40%, differences in their sequences. This means that over a length of 10 nucleotides at least 2 nucleotides are different, and 4 nucleotides are different over a length of 20 nucleotides, for example. Sequence identities, or differences between 2 sequences are preferably determined using the BLAST software, which program is publicly available on the website of the National Center for Biotechnology Information, the National Library of Medicine, at the National Institute of Health.

In an embodiment, a certain sequence can be determined as a Box sequence for a certain use. For example, if human mRNA is to be amplified in a method according to the invention, the described low homology may be determined by comparison with a human database or hybridizing human RNA with the Box sequence in a Northern Blot. If plant mRNA is to be amplified in the method according to the invention, the described low homology may be determined by comparison with plant ribonucleic acids. In an embodiment, a sequence suitable as a Box sequence in a certain use is not suitable as Box sequence in a different use. In another embodiment, a sequence suitable as a Box sequence in a certain use according to the present invention is useful as Box sequence in a different use.

In various embodiments, a Box sequence is preferably selected not to contain viral sequences, coding sequences, regulatory sequences (promoter or terminator sequences), or any other combination of such sequences, of viruses or bacteriophages.

In an embodiment of the present invention, use of a primer comprising a suitable Box sequence is highly advantageous, because the production of amplification artifacts is drastically reduced.

In an embodiment, a Box sequence is located in the 5′ region of the primer used in step (c). In another embodiment, the primer further contains a sequence of 1 to 6 random nucleotides (N1-N6), wherein the use of a primer containing 3 random nucleotides is especially preferred. In another embodiment, a single primer may only contain a single sequence of 1 to 6 nucleotides. However, in other embodiments, a mixture of otherwise identical primers may contain a random sequence in this region, i.e., in this region the primer may have any nucleotide sequence.

In another embodiment, the primer further contains a defined trinucleotide sequence. In another embodiment, a defined trinucleotide sequence is defined by its ability to preferentially bind near the 3′ end of a nucleic acid. In an embodiment, a defined trinucleotide sequence is a nucleotide sequence of any 3 nucleotides that is defined by its ability to bind to an RNA template. In another embodiment, a defined trinucleotide sequence is a nucleotide sequence of any 3 nucleotides that bind preferentially to an mRNA molecule as compared with binding to other RNA molecules. In a further embodiment of the present invention, the presence of a specific trinucleotide sequence in a primer facilitates complete amplification of all mRNA sequences in the starting material—regardless of the nature of the starting material. In another embodiment, the presence of a specific trinucleotide sequence in a primer further results in a selective amplification of mRNAs as compared with other RNAs. In one embodiment, the defined trinucleotide sequence is TCT. In an embodiment of the present invention, incorporation of the defined trinucleotide sequence has the specific advantage of non-random primer elongation. Alternatively, a mix of different primers can be used, each containing a different 3′ terminal nucleotide sequence.

Preferably, a primer containing a Box sequence has a length of up to 40 nucleotides, a length of up to 30 nucleotides is especially preferred.

In an embodiment, a sequence identified as Box 1 sequence is the same as a Box 2 sequence. In another embodiment, a Box 1 sequence is a different sequence from a Box 2 sequence.

In an embodiment, a method of the present invention preferably produces a single-stranded RNA which has an inverse sense orientation (i.e., an antisense sequence) in relation to the RNA in the starting material. In an embodiment, the antisense sequence may be in whole or in part in relation to the RNA in the starting material. In various embodiments, the antisense sequence has an inverse sense orientation in relation to the RNA in the starting material with regard to 3 nucleotides, 5 nucleotides, 10 nucleotides, 20 nucleotides, 50 nucleotides, 100 nucleotides, 200 nucleotides, 500 nucleotides, more than 500 nucleotides, or all nucleotides.

In an embodiment, removal of the RNA, such as in step (b), can be carried out by any of the methods for removal of RNA known in the art. In various embodiments, the RNA is hydrolyzed using an RNase, such as RNase I, RNase H, or both RNase I and RNase H. Any RNA, including, for example, rRNA and mRNA, may be removed. In an embodiment, RNAs are removed regardless of type of RNAs.

In an embodiment, the method of the present invention may be used for the amplification of bacterial mRNA, for the amplification of eukaryotic mRNA, for the amplification of degraded mRNA, or for the amplification of any combination of such mRNAs.

In one aspect of the present invention, a reverse transcriptase is used as DNA polymerase. Further, dATP, dCTP, dGTP and dTTP may be used as deoxyribonucleoside triphosphate monomers and ATP, CTP, GTP and UTP may be used as ribonucleoside triphosphate monomers.

In an embodiment, separating a double-stranded DNA into single-stranded DNAs can be accomplished by any techniques known in the art. In an embodiment, the DNA double strands can be separated into single strands using heat.

In an embodiment a single-stranded primer used in step (e) of various methods of the present invention comprises the sequence of an RNA polymerase promoter, which may be the promoter sequence for any known RNA polymerase such as T7, T3 or SP6 RNA polymerase.

Various embodiments of the method of the present invention enable highly specific amplification of mRNAs, preferably the mRNA present in a starting material is amplified by a factor (“amplification factor”) of at least 500 or at least 1000. Further, in various embodiments, the mRNA sequences present in a starting material are enriched, e.g., from 1% to 5% in the input RNA to at least 90% in the amplified RNA.

In another aspect of the present invention, the methods for amplification comprise the following further steps for amplification of ribonucleic acids after step (f):
(g) using the single-stranded RNAs generated in step (f) as template, single-stranded DNA is synthesized using reverse transcriptase, a single-stranded primer, comprising the Box 2 sequence, an RNA-dependant DNA polymerase and deoxyribonucleoside triphosphates;
(h) the RNA is removed;
(i) using the single-stranded DNA generated in (h) as template, double-stranded DNA is synthesized using a single-stranded primer, comprising a 5′—Promoter—Box 1 sequence—3′, a DNA polymerase and deoxyribonucleoside triphosphates;
(j) a multitude of single-stranded RNAs is synthesized using an RNA polymerase and ribonucleoside triphosphates.

Again, the RNA in step (h) may be hydrolyzed using an RNase.

In an embodiment of step (j) of the above method, single-stranded RNAs are produced which all have the inverse orientation.

In the methods of the present invention, mRNA is amplified and may be subjected to further analysis, such as analysis using microarrays. The analysis may be based on ribonucleic acids isolated from a biological sample. The analysis may also be based on ribonucleic acids obtained synthetically. In that context, the amount of mRNA, the sequence of mRNA, or both the amount and sequence of mRNA, in the starting material may be the subject of additional analysis.

In a further aspect, the present invention includes a kit. In another embodiment, a kit includes instructions on how to use the kit. In various embodiments, the kits described herein may be used for ribonucleic acid amplification according to the methods described herein. In an embodiment, a kit comprises the following components:
(a) a mixture of single-stranded primers comprising the following sequences 5′—Box 1 sequence—1 to 6 random nucleotides—a specific trinucleotide sequence—3′;
(b) a mixture of single-stranded primers comprising the following sequences 5′—Box 2 sequence—1 to 6 random nucleotides—a specific trinucleotide sequence—3′;
(c) a single-stranded primer comprising the following sequences 5′—promoter sequence—Box 1 sequence—3′;
(d) an RNA-dependent DNA polymerase;
(e) deoxyribonucleoside triphosphates;
(f) a DNA-dependent DNA polymerase;
(g) an RNA polymerase; and
(h) ribonucleoside triphosphates.

In an embodiment, a kit may comprise three different single-stranded primers. In another embodiment, a kit may further comprise a single-stranded primer, comprising the Box 2 sequence, an RNA-dependant DNA polymerase and deoxyribonucleoside triphosphates and a single-stranded primer, comprising a 5′—Promoter—Box 1 sequence—3′.

If the RNA is to be removed by RNase, the kit may, in addition to the above components, comprise RNase I, RNase H, or both RNase I and RNase H. The DNA polymerase mentioned above may be a reverse transcriptase and the kit may further comprise a T7 RNA polymerase. In a further embodiment, the kit also comprises a composition for labeling of DNA with a detectable moiety, a DNA-microarray or both a detectable moiety and a DNA-microarray.

Kits containing components for carrying out methods in accordance with the present invention are commercially available from AmpTec, Germany. Respective kits are sold under the trademark ExpressArt® Bacterial mRNA Amplification Kit or ExpressArt® Trinucleotide mRNA Amplification Kit for severely degraded RNA. The package inserts for these kits are herein incorporated by reference in their entireties.

### EXAMPLES

The following examples illustrate the use of the methods of the present invention for the amplification of mRNAs. The examples are based upon package inserts sold with the ExpressArt® Bacterial mRNA Amplification Kits from AmpTec, Germany. Similar kits have been sold as Trinucleotide mRNA Amplification Kit for severely degraded RNA and both package inserts (the package inserts of catalogue no. 8093-A12 and 8097-A12 of AmpTec catalogue 2005) are fully incorporated herein by reference in their entireties for all purposes.

### Example 1

This example provides one illustrative set of reagents for carrying out a universal method for selective amplification of mRNAs.

Reagents are provided in two kit boxes—Kit box I and Kit box II. The materials are provided for 12×2-rounds RNA amplifications.

Contents of Kit box I include:

| | | |
|---|---|---|
| Tube 1: Primer TR | 22.5 | μl |
| Tube 2: dNTP-Mix | 60.0 | μl |
| Tube 3: DEPC-H2O | 1500 | μl |
| Tube 4: 5x RT Buffer | 120.0 | μl |
| Tube 5: RNase Inhibitor | 30.0 | μl |
| Tube 6: RT Enzyme | 30.0 | μl |
| Tube 7: RNase | 30.0 | μl |
| Tube 8: Primer B | 15.0 | μl |
| Tube 9: 5x Extender Buffer | 225.0 | μl |
| Tube 10: Extender Enzyme A | 15.0 | μl |
| Tube 11: Primer Erase (Enzyme) | 30.0 | μl |
| Tube 12: Primer C | 150.0 | μl |
| Tube 13: Extender Enzyme B | 30.0 | μl |
| Tube 14: Carrier DNA | 90.0 | μl |
| Tube 15: Precipitation Carrier (Pellet Paint ®) | 90.0 | μl |
| Tube 16: Sodium Acetate (3M, pH 5) | 450.0 | μl |
| Tube 17: Solubilization Buffer (10 mM Tris-HCl, pH 8) | 240.0 | μl |
| Tube 18: NTP-Mix | 240.0 | μl |
| Tube 19: 10x Transcription Buffer | 60.0 | μl |
| Tube 20: RNA Polymerase | 60.0 | μl |
| Tube 21: DNase I | 30.0 | μl |
| Tube 22: Primer D | 30.0 | μl |
| Tube 23: Positive Control RNA | 12.5 | μl |
| Tube 24: Reaction Additive (DMSO) | 30.0 | μl |

Contents of Kit box II include:

| | | |
|---|---|---|
| cDNA Purification Spin Columns | 24 | pcs |
| Collection Tubes | 24 | pcs |
| Binding Buffer | 12 | ml |
| Washing Buffer | 8 | ml |
| Elution Buffer | 10 | ml |

Immediately upon arrival, all reagents of Kit box I are stored at −20° C. Repeated freeze thawing is to be avoided. The contents of Kit box II are stored at room temperature. Reagents are typically stable for 6 months, as may be verified by the expiration date on the kit box.

Additional materials include RNeasy MiniKit (Qiagen®, Valencia, Calif.), Eppendorf® or Gilson® 0.5-2 μl pipettes, RNase-free pipette-tips, RNase-free reaction tubes (0.5/1.5 ml), 100% ethanol and 70% ethanol, a microcentrifuge, and a commercially available thermocycle nucleotide amplifier (commonly known as a thermocycler).

Reactions, apart from the overnight in vitro transcription (see below), could be performed in a standard thermocycler (with the lid temperature adjusted to 110° C.). An air incubator is recommended for performing overnight in vitro transcription reactions at 37° C. Alternatively, a thermocycler with adjustable heating lid may be used (lid temperature adjusted to 45° C.). Optionally, a hybridization oven is used.

Positive control: The bacterial mRNA amplification kit contains E. coli total RNA as positive control. Two μl (100 ng) of Positive Control RNA (Tube 23) are used per kit reaction. The remainder of the positive control is stored at −80° C.

Chemical hazards: The Binding Buffer in Kit box II contains guanidine thiocyanate, which is harmful in contact with skin when inhaled or swallowed. Guanidine thiocyanate also liberates toxic gas, when mixed with strong acids. Always store and use the Binding Buffer away from food. Always wear gloves, and follow standard safety precautions during handling and make sure to comply with the safety rules of your laboratory.

Quality control: Components of the kitare tested in an amplification using the Positive Control RNA (Tube 23). Reagents are tested for the absence of nuclease activity.

For good quality eukaryotic total RNA samples, the standard ExpressArt® mRNA Amplification Kits are available: an oligo-dT primer anneals with the 3′-Poly(A) tail of intact eukaryotic mRNAs. For bacterial mRNAs, the ExpressArt® Bacterial mRNA amplification kits has been developed. Instead of oligo-dT, the first cDNA synthesis is performed with a special TRinucleotide primer (5′—Box-Random—3′-Trinucleotide-primer) that results in preferential priming near the 3′-end of any nucleic acid.

Very low priming is observed for rRNA. In addition, no loss in signal intensity and no loss in presence calls have been observed. There is also no need to remove rRNAs because there is less than 2% rRNAs in amplified RNAs. This new technology enables specific amplification of bacterial mRNAs.

The ExpressArt® Kits provide a highly sensitive and reproducible technology for linear mRNA amplification, as well as RNA isolation, in combination with microarray hybridization.

Various exemplary advantages of ExpressArt® mRNA Amplification include:

Special kits for selective amplification of Bacterial mRNAs;

Special kits for severely degraded RNAs;

Selective mRNA amplification and full sequence recovery;

No primer derived artifacts;

cDNA synthesis is uncoupled from insertion of T7-promoter;

With other systems, the frequently observed large amounts of template-independent high molecular weight amplification artifacts often limit the amplification of low or very low amounts of input RNA. With ExpressArt®, the “no-template-control” is observed to be free of any amplified background, even after two and three rounds of amplification. This enables the amplification of sub-nanogram amounts of input total RNA, as demonstrated by the amplification of RNA from 4-cell embryos of C. elegans (Baugh et al. 2003);

Various other exemplary advantages of ExpressArt® mRNA Amplification include:

No continuous shortening with loss of mRNA sequences;

dsDNA with “TRinucleotide primer” (Box-random-trinucleotide primer) not with random primer;

Three amplification rounds as faithful as two;

Flexible transition between laser microdissection, cryosections, biopsies, etc.;

No need for careful control of input RNA amounts. Small and large amounts can be directly compared, regardless if they require two or three amplification rounds;

Rescue of drop-outs in series with two amplification rounds by performing a third round, but only performed for the samples with insufficient yields;

Improved detection;

Hundreds of additional genes amplified above expression threshold and many additionally identified differentially expressed genes;

Archives of templates;

Simple and easy re-evaluation of old samples in new contexts, with changed microarray designs;

Amplified RNAs contain defined sequences at both ends; and

Faithful reproduction of dynamic gene expression levels

### Example 2

Highly reproducible array hybridizations can be performed with a few cells, e.g., individual 4-cell embryos of C. elegans (Baugh et al. 2003).

Historically, a linear, isothermal amplification strategy based on in vitro transcription with T7 RNA-polymerase was used (Van Gelder et al. 1990; Eberwine et al. 1992). In this procedure, mRNA was converted into double-stranded cDNA, using a T7-promoter/oligo-dT primer for first strand cDNA-synthesis and limited RNase H digestion for self-priming during second strand synthesis. For amplification, these dsDNA-molecules were used as templates for in vitro transcription, for example, resulting in linear amplification maintaining the expression patterns of the original mRNAs (Poirier et al. 1997; Puskas et al. 2002).

A number of problems have been observed with this approach, including, for example:
(i) amplified RNA (aRNA) was 3′-biased since transcription and cDNA-synthesis with the T7-promoter/oligo-dT primer start at the poly(A)-tail of the original mRNA;
(ii) a second amplification was based on random priming, causing reduction of fragment length, which was even more pronounced when only small amounts of input RNA were available;
(iii) the use of the T7-promoter/oligo-dT primer in the first cDNA-synthesis could lead to large amounts of non-template high molecular weight artifacts, which became dominant with low amounts of input RNA (Baugh et al. 2001);
(iv) only high quality RNA samples with intact RNA could be used; and
(v) selective amplification of bacterial mRNAs was not achieved.

The ExpressArt® mRNA Amplification Kits of the present invention provide a technology which addresses these problems. With a special TRinucleotide mRNA amplification kit, the intact mRNA as well as all mRNA fragments are converted to cDNA with a special “TRinucleotide primer” (Box-1-random-trinucleotide primer; without T7-promoter). Also based on TRinucleotide primer technology, selective amplification of bacterial mRNAs is possible. The TRinucleotide primer permits preferential priming near the 3′-ends of all nucleic acid molecules. A model experiment illustrates its performance.

To minimize further 3′-bias in the next step, double-stranded cDNA is generated with a second “TRinucleotide primer” (Box-random-trinucleotide primer), again with preferential priming near the 3′-ends of the cDNAs. This results in the generation of almost full-length double-stranded cDNAs.

After denaturation, the second cDNA strand is primed in reverse orientation, using a T7-promoter/Box-1 primer. This leads to double-stranded cDNA with a functional T7-promoter at one end and the Box sequence tag at the other end. This dsDNA product is used as template for in vitro transcription, generating amplified, antisense oriented RNA with defined sequences at both ends.

This is an advantage for second and especially for third round amplifications, where size reductions of amplified RNAs are avoided. This enables the comparison of samples that contain very divergent amounts of input RNA.

Now, it is not only possible to perform highly reproducible array hybridizations with a few cells, e.g., individual 4-cell embryos of C. elegans (Baugh et al. 2003), even severely degraded RNAs yield excellent results.

For a sample of literature, see:

Baugh L R, Hill A A, Brown E L, Hunter C P (2001). Quantitative analysis of mRNA by in vitro transcription. Nucleic Acids Res. 29:E29;

Baugh L R, Hill-Harfe K, Brown G, Hunter C P (2003), personal communication;

Boularand S, Darmon M C, Mallet J (1995). The human tryptophan hydroxylase gene: an unusual complexity in the 5′-untranslated region. J Biol Chem. 270: 3748-3756;

Eberwine J, Yeh H, Miyashiro K, Cao Y, Nair S, Finell R, Zettel M, Coleman P (1992). Analysis of gene expression in single life neurons. Proc. Natl. Acad. Sci. 89: 3010-3014;

Mathieu-Daude F, Welsh J, Vogt T, McClelland M (1996). DNA rehybridization during PCR: the Cot effect and its consequences. Nucleic Acids Res. 24: 2080-2086;

Poirier F, Pyati J, Wan J S, Erlander M G (1997). Screening differentially expressed cDNA clones obtained by differential display using amplified RNA. Nucleic Acids Res. 25: 913-914;

Puskas L G, Zvara A, Hackler L, Van Hummelen P (2002). RNA amplification results in reproducible microarray data with slight ratio bias. BioTechniques 32:1330-1340;

Van Gelder R N (1990). Amplified RNA synthesized from limited quantities of heterogeneous cDNA. Proc. Natl. Acad. Sci. 87: 1663-1667;

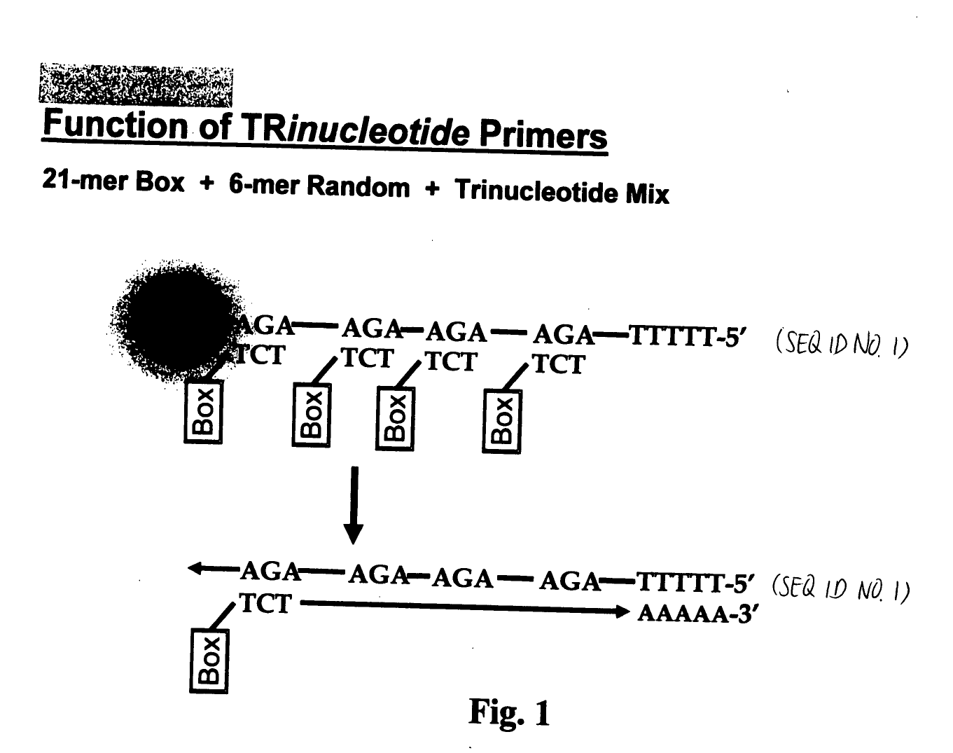

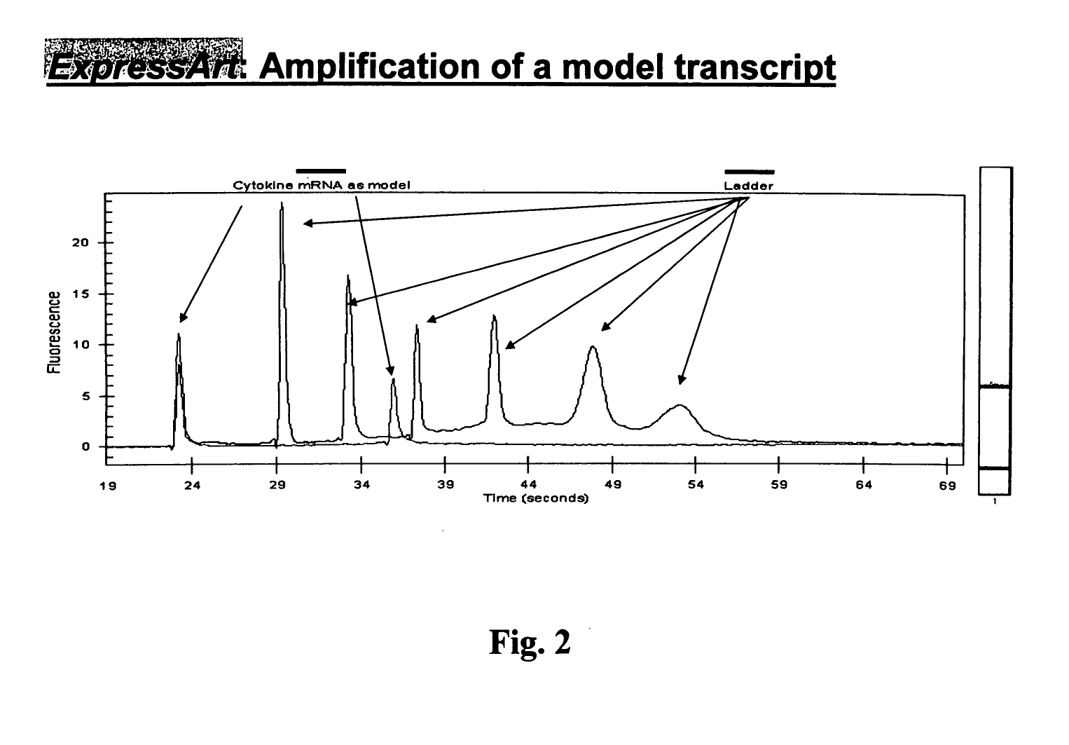

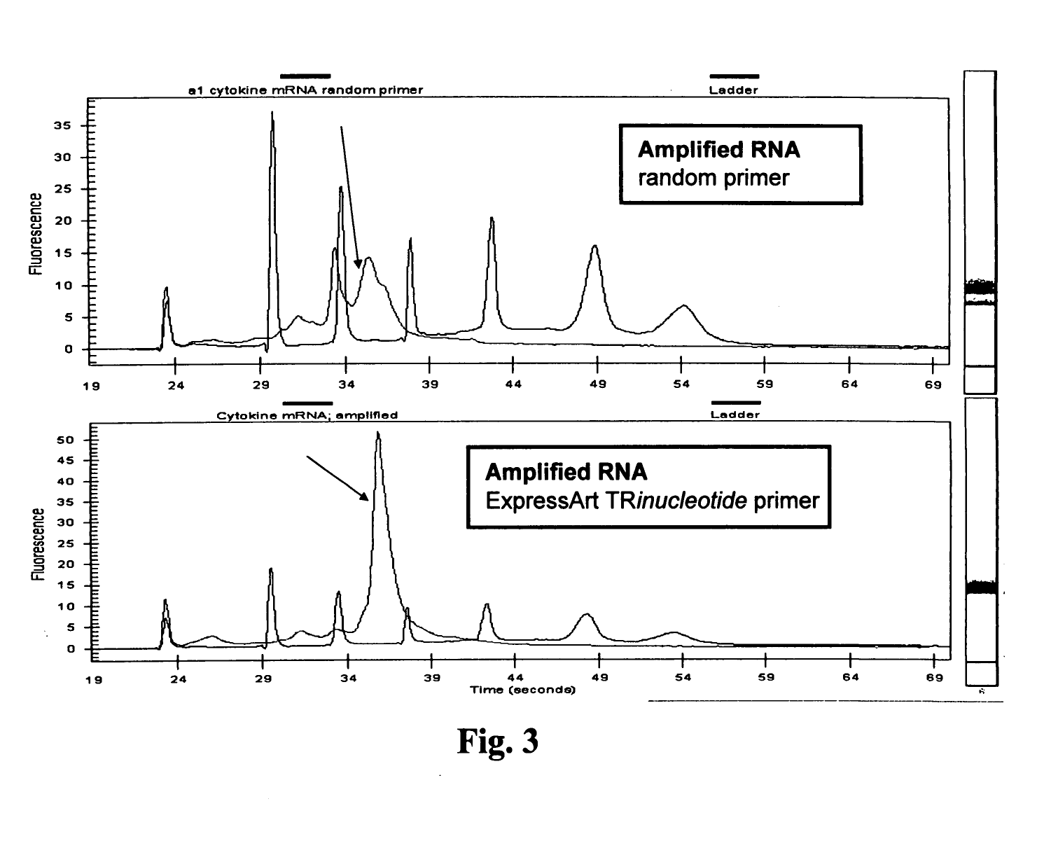

Model experiment to illustrate one of the unique properties of ExpressArt® TRinucleotide primers (FIG. 1): A defined in vitro transcript of 800 nucleotide length is used as input mRNA model (see, e.g., the top tracing in the electropherogram in FIG. 2). Amplification with ExpressArt® technology and the TRinucleotide primer (Box-random-trinucleotide primer) results in essentially full-length aRNA (the top tracing in bottom electropherogram in FIG. 3). For comparison, the same reaction steps are used, but the mix of 3′-terminal trinucleotide sequences in the TRinucleotide primer is replaced by a random trinucleotide. This results in a mixture of shorter aRNAs with a minor fraction (if any) of full-length product (see, e.g., the top tracing in top electropherogram in FIG. 3).

Before you start: The following may be useful to consider in performing methods of the present invention:

How to store and handle reaction tubes: do not autoclave; do not remove from bag by inserting your hand (not even with gloves); instead, pour onto fresh tissue on bench; never touch inside of cap when opening or closing.

How to store and handle pipette tips: do not autoclave; always replace pipette box cover after finishing work.

How to store and handle stock solutions: do not insert pipette; instead, pour small aliquot in tube; always replace cap after finishing work.

How to thaw liquids in small tubes: freezing generates concentration gradient instead of homogeneous solution; always mix thoroughly, e.g., by thawing on a Thermomixer (1000 rpm) or by inverting and flicking tube.

How to mix small volumes in reaction tubes: small enzyme volumes “precipitate” at the bottom of the tube; always mix by flicking tube or by pipette mixing the complete reaction volume.

How to perform ethanol precipitation: always proceeding in the order of RNA solution+(salt+carrier), mix thoroughly, then add ethanol; do not over dry pellet in speed vacuum; instead, air dry pellet.

How to use spin columns: do not touch surface of matrix; do not use collection tube and cap from last spin; instead, transfer eluate into fresh tube.

### Example 3

### Microarray Hybridization

RNA Quality Control: Historically linear mRNA amplification was limited to mRNAs with 3′-Poly(A) and required high quality RNA. Therefore, selective amplification of bacterial mRNAs was hindered. With the introduction of the ExpressArt® Bacterial mRNA amplification kits, this problem is addressed.

In addition to gel electrophoresis, the Agilent 2100 bioanalyzer combined with RNA 6000 Nano and Pico LabChips is widely used for high-resolution analysis of small and very small RNA samples. Expected electropherograms vary, depending on species, tissue type and method of RNA isolation. See Agilent Application Note “Stringent RNA Quality Control using the Agilent 2100 Bioanalyzer” (Krupp, 2004). For RNA isolation in the low nanogram and picogram range, use of the ExpressArt® PICO RNA CARE reagents is recommended.

Stringent RNA quality control may be useful to assure that fragmented rRNAs and other RNA aggregates are resolved and do not erroneously migrate as one band. This may be achieved by denaturing electrophoresis conditions, or simply by heating the RNA sample for 2 min at 70° C., immediately before performing native electrophoresis with a gel or with the Agilent 2100 bioanalyzer.

An improved general RNA quality assessment has been introduced, see, e.g., Agilent Application Note 5989-1165EN “RNA Integrity Number (RIN)—Standardization of RNA Quality Control” (Mueller, Lightfoot, Schroeder, 2004). A RIN value is derived from the RNA profiles in electropherograms, with a range of 1 to 10 and with RIN=10 for the highest RNA quality.

Electropherograms of amplified Bacterial mRNAs: E. coli total RNA samples (200 ng) are amplified with the ExpressArt® Bacterial mRNA Amplification kit. After the first round, approximately 5 μg aRNAs are obtained and aliquots (10%) are used in the second amplification round, yielding approximately 50 μg aRNAs with a medium length of 500 nucleotides.

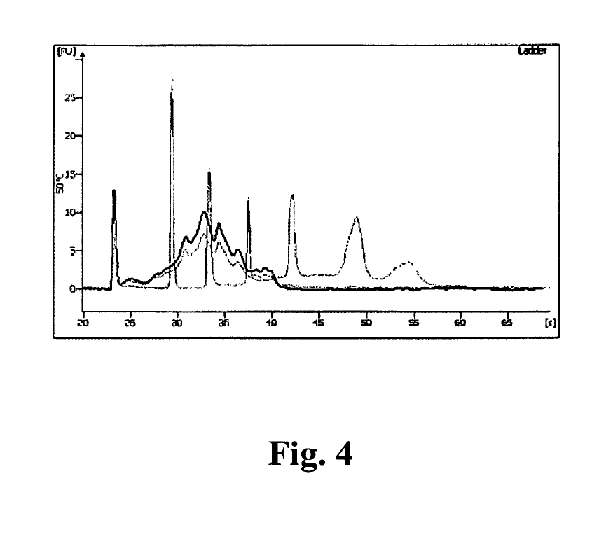

Electropherograms of amplified bacterial mRNAs are obtained after two ExpressArt amplification rounds (FIG. 4). Three RNA profiles are shown, one profile for the RNA ladder, and two profiles of amplified RNAs. The top profile is obtained with amplified RNAs using total RNA from E. coli cultured at 50° C., the lower profile from E. coli cultured at 37° C.

General characteristics of microarray hybridization data: Biotinylated, amplified RNAs are hybridized on Affymetrix E. coli Genome 2.0 GeneChips.

In standard hybridizations, high stringency conditions are used because labeled cDNAs (obtained with random primers) contain a high fraction of rRNAs. In contrast, the ExpressArt® kits enable specific amplification of bacterial mRNAs and the same hybridization conditions are used as for specifically amplified eukaryotic Poly(A) mRNAs.

The observed high numbers of presence calls and high signal-background ratios show the appearance of complete and specific amplification of E. coli mRNAs, in an example: 3938 or 38.6% (growth at 37° C.), 4728 or 46.3% (growth at 50° C.), with signal-background ratios of 45 to 50, scale factors of 10 to 11, and average signals (P) >4,000.

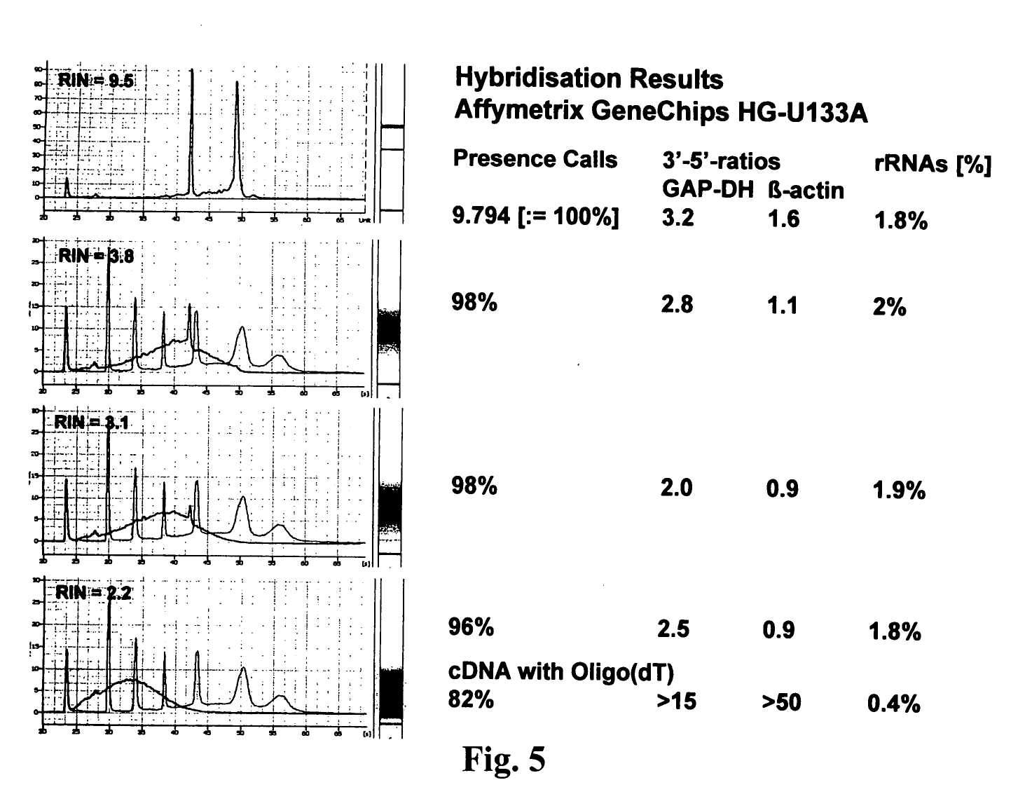

Amplification of rRNAs: The 16S and 23S rRNAs are the bulk of total RNA samples (>80%). Very stringent hybridization conditions are used for non-selective labeling or amplification and can result in low detection sensitivity. With ExpressArt® Bacterial mRNA amplification kits, a strong selection against rRNA amplification leads to very low hybridization signals for rRNA, and the total amount of rRNA in amplified RNAs is below 2% (FIG. 5).

Differential gene expression: Reliable detection of differentially expressed gene expression is evaluated with E. coli heat shock as model experiment. RNA samples are compared, obtained after growth at 37° C. versus 50° C.

Induced and repressed genes are listed in the following tables, comparing

i) published data: Richmond C S, Glasner J D, Mau R, Jin H, Blattner F R (1999). Genome-wide expression profiling in Escherichi coli K12. Nucleic Acids Res. 27: 3821-3835.

ii) data with direct fluorescent cDNA labeling, using 50 μg of E. coli RNAs and random primers for the reverse transcription reaction, followed by hybridizations on MWG E. coli K12 Arrays.

iii) data with ExpressArt® Bacterial mRNA amplification, using 200 ng of E. coli input total RNAs and indirect fluorescent labeling of antisense aRNAs, using the ExpressArt® Amino-Allyl Bacterial mRNA amplification kit. Hybridizations are performed with MWG E. coli K12 Arrays and stringent hybridization conditions.

iv) data with ExpressArt® Bacterial mRNA amplification, using 200 ng of E. coli input total RNAs, and generating biotinylated aRNAs with the Enzo High Yield RNA Labelling Kit. Hybridizations are performed on Affymetrix E. coli Genome 2.0 GeneChips, but using the standard conditions for biotinylated eukaryotic antisense mRNAs.

Apart from correct assignments, also quantitative fold changes are very similar, comparing hybridizations on the same array format, i.e., the MWG Arrays using fluorescent-labeled cDNAs without amplification and fluorescent-labeled aRNAs after ExpressArt® Bacterial mRNA amplification (close similarities are indicated as shaded areas in the following tables).

Examples for genes induced by heat shock

| Gene | Gene description | Richmond et al., 1999 | 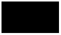 | 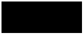 | ExpressArt ®& Affymetrix |
|---|---|---|---|---|---|
| clpb | heat shock protein | medium | medium | high | high |
| dnaj | chaperone with dnak, heat shock protein | high | 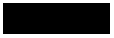 | 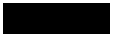 | medium |
| dnak | chaperone hsp70, dna biosynthesis, heat shock protein | medium | 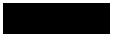 | 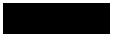 | low |
| grpe | phage lambda replication, | medium | low | medium | medium |
| | host dna synthesis, | | | | |
| | heat shock protein | | | | |
| hslj | heat shock protein | medium | low | low | medium |
| hslu | heat shock protein | low | low | low | low |
| | hslvu, atpase subunit | | | | |
| hslv | heat shock protein hslvu, | medium | low | medium | medium |
| | proteasome-related | | | | |
| | peptidase sub-unit | | | | |
| htpx | integral membrane protein, heat shock protein | medium | 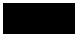 | 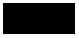 | medium |
| ibpa | heat shock protein | high | high | 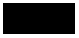 | high |
| ibpb | heat shock protein | high | high | high | high |
| lon | dna-binding, atp- | medium | low | medium | low |
| | dependent protease la, | | | | |
| | heat shock k-protein | | | | |

Examples for genes repressed by heat shock

| | | Richmond | cDNA | ExpressArt ® | ExpressArt ® |
|---|---|---|---|---|---|
| Gene | Gene description | et al., 1999 | & MWG | & MWG | & Affymetrix |
| suca | 2-oxoglutarate dehydrogenase | medium | high | high | high |
| | (decarboxylase component) | | | | |
| sucb | 2-oxoglutarate dehydrogenase (dihydrolipoyltranssuccinase e2 component) | medium | high | 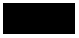 | high |
| icda | isocitrate dehydrogenase, | medium | low | medium | medium |
| | specific for nadp+ | | | | |
| atpa | membrane-bound atp synthase, | high | low | low | low |
| | f1 sector, alpha-subunit | | | | |
| atpg | membrane-bound atp synthase, | high | low | low | low |
| | f1 sector, gamma-subunit | | | | |
| atph | membrane-bound atp synthase, | high | low | low | low |
| | f1 sector, delta-subunit | | | | |
| nuoh | nadh dehydrogenase I chain H | medium | low | medium | medium |
| nuom | nadh dehydrogenase I chain M | high | 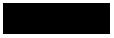 | 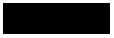 | medium |
| cyoa | cytochrome o ubiquinol oxidase subunit II | low | 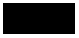 | 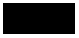 | high |
| cyob | cytochrome o ubiquinol oxidase subunit I | low | 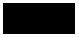 | 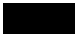 | high |
| tolb | periplasmic protein involved in the tonb- independent uptake of group a colicins | medium | 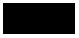 | 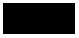 | low |

### Example 4

### Protocol for the Bacterial mRNA Amplification Kit (Nano Version)

The NANO version of the ExpressArt® Bacterial mRNA amplification kits is suitable for a wide range, from 5 ng to 700 ng of input total RNA. According to the amount of input total RNA and the required yields of aRNA, it can be used for 1-round (aRNA yields in the low μg range) or 2-rounds (aRNA yields in low and high μg range).

A: First Round Amplification

A1. First Strand cDNA Synthesis

To the extent possible, starting RNA is free of any genomic DNA. The Bacterial mRNA amplification kits are extremely sensitive to contaminating DNA fragments. A DNase treatment should be combined with a spin column purification to remove as many fragments of digested DNA as possible.

General guidelines in section “Before you start” described in Example 1 are observed.

A thermocycler is programmed with the temperatures and times, given in this protocol. See, e.g., “Thermocycler profiles”.

Total RNA ranges from 5 ng to 700 ng.

Optionally, instead of 5 μl RNA, up to 11.5 μl RNA is used. To maintain total reaction volumes, omit H₂O in Mix 1 and Mix 2 described below.

If running more than one reaction at a time, Master Mixes are prepared.

To check amplification performance, a reaction tube containing 2 μl Positive Control RNA (100 ng, Tube 23) is processed in parallel.

Optionally, to check for primer-derived artifacts, a reaction containing 5 μl DEPC-H2O (Tube 3) instead of RNA is also processed in parallel.

First Strand cDNA Synthesis Mix 1 is prepared according to the table below. An appropriate Master mix volume is used for processing multiple samples.

First Strand cDNA Synthesis Mix 1

| | | |
|---|---|---|
| DEPC-H2O | Tube 3 | 3.0 μl |
| dNTP-Mix | Tube 2 | 1.0 μl |
| Primer TR | Tube 1 | 1.0 μl |

Five μl Mix 1 is added to 5 μl of each RNA (and to the optional negative control). The mixtures are incubated 4 minutes at 65° C. in a thermocycler (with heating lid, use standard setting, e.g., 110° C.). Samples are cooled to 37° C. In the meantime, First Strand cDNA Synthesis Mix 2 is prepared at room temperature.

First Strand cDNA Synthesis Mix 2

| | | |
|---|---|---|
| DEPC-H2O | Tube 3 | 4 μl |
| 5x RT Buffer | Tube 4 | 4 μl |
| RNase Inhibitor | Tube 5 | 1 μl |
| RT Enzyme | Tube 6 | 1 μl |

The First Strand cDNA Synthesis Mix 2 (10 μl) is added to each sample and mixed well by gently flicking the tube. The samples are incubated in a thermocycler according to the following conditions: 37° C./45 min; 45° C./10 min; 50° C./5 min; 37° C./1 min.

Primer Erase Mix 6 is prepared according to the table below.

Primer Erase Mix 6

| | | |
|---|---|---|
| DEPC-H2O | Tube 3 | 3 μl |
| 5x Extender Buffer | Tube 9 | 1 μl |
| Primer Erase | Tube 11 | 1 μl |

Five μl of Primer Erase Mix 6 is added to each sample and incubations are continued according to the following conditions: 37° C./5 min; 80° C./15 min; 37° C./1 min.

A2. RNA Removal

RNase Mix 3 is prepared according to the table below.

RNase Mix 3

| | | |
|---|---|---|
| DEPC-H2O | Tube 3 | 3 μl |
| 5x Extender Buffer | Tube 9 | 1 μl |
| RNase | Tube 7 | 1 μl |

Five μl of RNase Mix 3 is added to 25 μl of First Strand cDNA Reaction from A1. The mixture is incubated for 20 minutes at 37° C.

A3.Second Strand cDNA Synthesis

Second Strand cDNA Synthesis Mix 4 is prepared according to the table below.

Second Strand cDNA Synthesis Mix 4

| | | |
|---|---|---|
| DEPC-H2O | Tube 3 | 10 μl |
| 5x Extender Buffer | Tube 9 | 3 μl |
| Primer B | Tube 8 | 1 μl |
| dNTP-Mix | Tube 2 | 1 μl |

Fifteen μl of Mix 4 is added to each First Strand cDNA Synthesis Reaction from A2 and incubated as follows in a thermocycler: 96° C./1 min; 37° C./1 min.

Extender Enzyme A Mix 5 is prepared according to the table below.

Extender Enzyme A Mix 5

| | | |
|---|---|---|
| DEPC-H2O | Tube 3 | 3 μl |
| 5x Extender Buffer | Tube 9 | 1 μl |
| Extender Enzyme A | Tube 10 | 1 μl |

Five μl of Extender Enzyme A Mix 5 is added to each sample and mixed well by gently flicking the tube. Continue the incubation at 37° C./30 min.

Primer Erase Mix 6 is prepared according to the table below.

Primer Erase Mix 6

| | | |
|---|---|---|
| DEPC-H2O | Tube 3 | 3 μl |
| 5x Extender Buffer | Tube 9 | 1 μl |
| Primer Erase | Tube 11 | 1 μl |

Five μl of Primer Erase Mix 6 is added to each sample and the samples are mixed well by gently flicking the tube. Continue the incubation according to the following conditions: 37° C./5 min; 96° C./6 min; 37° C./1 min.

Five μl of Primer C (Tube 12) is added to each sample and mix well by gently flicking the tube. Incubation is continued in a thermocycler using the following conditions: 96° C./1 min, followed by 37° C./1 min.

Extender Enzyme B Mix 7 is prepared according to the table below.

Extender Enzyme B Mix 7

| | | |
|---|---|---|
| DEPC-H2O | Tube 3 | 2 μl |
| 5x Extender Buffer | Tube 9 | 2 μl |
| Extender Enzyme B | Tube 13 | 1 μl |

Five μl of Extender Enzyme B Mix 7 is added to each sample and the samples are mixed well by gently flicking the tube. Incubation is continued according to the following conditions: 37° C./30 min; 65° C./15 min; 37° C./1 min. The tubes are spun briefly to collect liquid.

A4. cDNA Purification using Spin Columns

Thirty-two ml 100% ethanol is added to 8 ml stock solution of Washing Buffer (Kit box II) and the tubes are mixed well.

Also see, e.g., “How to handle Spin Columns” in section “Before you start” in Example 1.

Purification Mix 8 is prepared according to the table below.

Purification Mix 8

| | | |
|---|---|---|
| Binding Buffer | (box II) | 350 μl |
| Carrier DNA | Tube 14 | 3 μl |

Three hundred and fifty-three μl of Mix 8 is added to each Second Strand cDNA Reaction from A3. cDNA Purification Spin Columns are inserted into Collection Tubes. The sample is pipetted onto each column and the columns are centrifuged for 1 min at maximum speed in a table top centrifuge. Note that guanidine thiocyanate in the Binding Buffer is an irritant. Always wear gloves and follow standard safety precautions to minimize contact when handling.

The flow-through is discarded and the columns are re-inserted into the same Collection Tubes. Five hundred μl Washing Buffer (with Ethanol added) is added to the columns and the columns are centrifuged for 1 min at maximum speed. The flow-through is discarded and the columns are re-inserted in the same Collection Tubes and washed with 200 μl Washing Buffer. The columns are centrifuged for 1 min at maximum speed. The flow-through and the Collection Tubes are discarded.

The columns are inserted into fresh 1.5 ml reaction tubes and 50 μl of Elution Buffer is added to the columns. The Elution Buffer is pipetted in the middle of the columns, directly on top of the matrix, without disturbing the matrix with the pipette tip. The columns are incubated for at least 1 min, then centrifuged for 1 min at maximum speed. The columns are eluted a second time with 50 μl Elution Buffer into the same reaction tube, incubated for at least 1 min and centrifuged again for 1 min at maximum speed. Eluate is transferred to fresh reaction tube for further processing.

A5. Ethanol Precipitation of the Purified cDNA

The Precipitation Carrier (Tube 15) is stored in the dark. For long-term storage, the Precipitation Carrier tubes are kept at −20° C. Smaller aliquots are kept at 4° C. for about 1 month.

Precipitation Mix 9 is prepared according to the table below.

Precipitation Mix 9

| | | |
|---|---|---|
| Sodium Acetate | Tube 16 | 10 μl |
| Precipitation Carrier | Tube 15 | 2 μl |

Twelve μl of Mix 9 is added to each eluate (100 μl; from A4) and the tubes are mixed well. 220 μl of 100% ethanol (room temperature) is added, the tubes are mixed again, and incubated for 2 min at room temperature. The cDNA is centrifuged at maximum speed for 10 min at room temperature. The supernatant is discarded and the pink-colored pellet is washed with 200 μl of 70% ethanol (room temperature). The tubes are centrifuged for 5 min at maximum speed and the supernatant is removed with a pipette.

To ensure that as much liquid as possible is removed, the pellet is spun briefly to collect liquid, and all remaining liquid is removed with a pipette tip. The pellets are air dried by leaving the tubes open for about 5 min at room temperature, covered with fresh tissue paper. The pellets are not to be dried in a speed vacuum. The pellet is dissolved in 8 μl Solubilization Buffer (Tube 17) and kept at room temperature for further amplification. Alternatively the samples are stored at −20° C. for later use.

A6. Amplification via in vitro Transcription

For labeling and microarray hybridization after one amplification round, examples are provided in section B6.

In vitro Transcription Mix 10 is prepared according to the table below.

In vitro-Transcription Mix 10

| | | |
|---|---|---|
| NTP-Mix | Tube 18 | 8 μl |
| 10x Buffer | Tube 19 | 2 μl |
| RNA Polymerase | Tube 20 | 2 μl |

Using 0.5 ml RNase-free PCR tubes, the in vitro-Transcription Mix is prepared by adding the components in the given order. Work is done at room temperature because on ice spermidine in the buffer can cause precipitation of DNA template.

Twelve μl in vitro-Transcription Mix 10 is added to 8 μl cDNA from A5. The transcription is incubated overnight at 37° C. in a thermocycler with heating lid adjusted to 45° C.; or preferentially in a hybridization oven. A thermocycler WITHOUT adjustable heating lid is not to be used because high lid temperature (usually >100° C.) of non-adjustable heating lid could negatively affect the efficiency of the transcription reaction. One μl DNase I (Tube 21) is added to each reaction and the mixtures are incubated further at 37° C. for 15 min.

A7. RNA-Purification using RNeasy Mini Kit (Qiagen®, not Provided with the ExpressArt® Kit)

4 volumes of 100% ethanol are added to RPE buffer, as indicated on the bottle. aRNA Purification Mix 11 is prepared according to the table below.

aRNA Purification Mix 11

| | |
|---|---|
| RNase-free water | 80 μl |
| RLT (Lysis Buffer) | 350 μl |

The purification columns are inserted into the collection tubes. 430 μl of Mix 11 is added to each in vitro-Transcription Reaction. The mixtures are mixed thoroughly and 250 μl 100% ethanol is added. The mixtures are pipetted onto the column. The column is centrifuged for 15 sec at 10,000 rpm in a table top centrifuge.

The flow-through is discarded and the columns are re-inserted into the same collection tubes. 500 μl RPE Buffer (with ethanol added) is added to the columns and the columns are centrifuged as above. The flow-through is discarded, the columns are re-inserted into the same collection tubes and washed with 500 μl RPE Buffer. The columns are centrifuged for 2 min. The flow-through is discarded, the collection tubes are re-inserted and centrifuged for 1 min at maximum speed to get rid of residual RPE Buffer.

The columns are inserted in new 1.5 ml RNase-free reaction tubes and 50 μl of RNase-free water is added to the columns. The water is pipetted in the middle of the columns, without disturbing the matrix with the pipette tip. The columns are incubated for 1 min and centrifuged for 1 min at 10,000 rpm. The columns are eluted a second time with 50 μl RNase-free water in the same collection tube, incubated for 1 min, and centrifuged again for 1 min at 10,000 rpm. Eluate is transferred to fresh RNase-free reaction tube for further processing.

A8. Ethanol Precipitation of the Purified aRNA

The Precipitation Carrier (Tube 15) is stored in the dark. For long-term storage, the tube is kept at −20° C. Smaller aliquots are kept at 4° C. for about 1 month.

Precipitation Mix 9 is prepared according to the table below.

Precipitation Mix 9

| | | |
|---|---|---|
| Sodium Acetate | Tube 16 | 10 μl |
| Precipitation Carrier | Tube 15 | 2 μl |

12 μl of Mix 9 is added to each eluate (100 μl from A7) and mixed well. 220 μl of 100% ethanol is added, the mixture is mixed again, and incubated for 2 min at room temperature. The cDNA is centrifuged at maximum speed for 10 min at room temperature.

The supernatant is discarded and the pink-colored pellet is washed with 200 μl of 70% ethanol (room temperature). The mixture is centrifuged for 5 min at maximum speed and the supernatant is removed with a pipette.

To ensure that as much liquid as possible is removed, the mixture is spun briefly to collect liquid, and the remaining liquid is removed with a pipette tip. The pellets are air dried by leaving the tubes open, but covered with fresh tissue paper, for about 5 min at room temperature. The pellets are not to be dried in a speed vacuum. The pellet is dissolved in 6 μl DEPC-H2O (Tube 3) and kept on ice.

A9. Control of aRNA Product Quantity and Quality

General suggestions for the second amplification round:

For input amounts of total RNA greater than 100 ng, 1 μl of aRNA from the first round (1 of the 6 μl obtained) is used. If lower amounts were used (with a minimum of 50 ng), then 2 μl of aRNA is used for second round amplification.

For product analysis: 1 μl of aRNA is used and 1 μl of water is added. 1 μl of diluted aRNA is used for Bioanalyzer and a second μl is used for photometric quantification. With 50-100 ng input total RNA, the total yield of aRNA is greater than 1 μg and 1 μl contains about 200 ng aRNA.

Photometric quantification: If 50-100 ng of input total RNA were used, 1 μl of the diluted aRNA is suitable for photometric quantification (dilution in up to 50 μl low salt buffer or water, measuring against a blank using the same buffer). With 50-100 ng of input total RNA, the yield of amplified RNA ranges between about 1-3 μg. If an additional second amplification round is required, 0.5 to 0.8 μg of amplified RNA is used (see section B).

Quality Control with Agilent 2100 bioanalyzer: Ionic compounds interfere with capillary electrophoresis. The signal may be significantly compressed by residual salt in the ethanol precipitate. If a broad size distribution is expected, the minimum recommended RNA concentration is 50-100 ng/μl (lower concentrations are possible for total RNA with its prominent rRNA peaks). The RNA size distribution is monitored with the bioanalyzer, but quantitation may indicate too low RNA amounts. Example electropherograms of two rounds amplified E. coli RNAs are shown in section “Electropherograms of amplified bacterial mRNAs” in Example 1.

B: Second Round Amplification

Amplified RNA is again reverse transcribed into cDNA to produce high yields of aRNA via a second round of amplification (see Expected yields). To obtain amplified labeled antisense RNA, the amplified DNA template (steps B4/B5) is used for in vitro transcription with an RNA labeling kit (see options in section B6).

B1. First Strand cDNA Synthesis

No more than 500-800 ng RNA from the first amplification round from step A8 is used.

First Strand Mix 12 is prepared according to the table below.

First Strand Mix 12

| | | |
|---|---|---|
| dNTP-Mix | Tube 2 | 1 μl |
| Primer D | Tube 22 | 2 μl |
| Reaction Additive | Tube 24 | 2 μl |

5 μl of Mix 12 is added to 5 μl RNA (500-800 ng; see section A9) from the first amplification round from step A7. The mixture is incubated for 4 min at 65° C. in a thermocycler (with heating lid, use standard temperature setting, e.g., 110° C.), then the samples are immediately cooled to 45° C.

The First Strand cDNA Synthesis Mix 2 is prepared according to the table below, at room temperature.

First Strand cDNA Synthesis Mix 2

| | | |
|---|---|---|
| DEPC-H2O | Tube 3 | 4 μl |
| 5× RT Buffer | Tube 4 | 4 μl |
| RNase Inhibitor | Tube 5 | 1 μl |
| RT Enzyme | Tube 6 | 1 μl |

10 μl of Mix 2 is added to each sample, the mixture is incubated in the 45° C. hot thermocycler. The mixture is mixed well by gently flicking the tube. Incubation is continued in a thermocycler according to the following conditions: 45° C./30 min, 70° C./15 min. The samples are immediately placed on ice.

B2. RNA Removal

RNase Mix 3 is prepared according to the table below.

RNase Mix 3

| | | |
|---|---|---|
| DEPC-H2O | Tube 3 | 3 μl |
| 5× Extender Buffer | Tube 9 | 1 μl |
| RNase | Tube 7 | 1 μl |

5 μl of RNase Mix 3 is added to 20 μl of First Strand cDNA Reaction from B1. The mixture is incubated for 20 minutes at 37° C.

B3. Second Strand cDNA Synthesis

Second Strand cDNA Synthesis Mix 13 is prepared according to the table below.

Second Strand cDNA Synthesis Mix 13

| | | |
|---|---|---|
| DEPC-H2O | Tube 3 | 10 μl |
| Primer C | Tube 12 | 5 μl |
| 5× Extender Buffer | Tube 9 | 4 μl |
| dNTP-Mix | Tube 2 | 1 μl |

20 μl of Mix 13 is added to each sample from B2, then the mixture is incubated according to the following conditions: 96° C./1 min, 37° C./1 min.

Extender Enzyme B Mix 14 is prepared according to the table below.

Extender Enzyme B Mix 14

| | | |
|---|---|---|
| DEPC-H2O | Tube 3 | 3 μl |
| 5× Extender Buffer | Tube 9 | 1 μl |
| Extender Enzyme B | Tube 13 | 1 μl |

5 μl of Extender Enzyme B Mix 14 is added to each sample and the mixture is mixed well by gently flicking the tube. The incubation continues according to the following conditions: 37° C./30 min, 65° C./15 min.

The samples are placed on ice and spun briefly to collect liquid.

B4. cDNA Purification using Spin Columns

32 ml 100% ethanol is added to the 8 ml stock solution of Washing Buffer (Kit box II) and the mixture is mixed well. Purification Mix 8 is prepared according to the table below.

Purification Mix 8

| | | |
|---|---|---|
| Binding Buffer | (box II) | 275 μl |
| Carrier DNA | Tube 14 | 3 μl |

278 μl of Mix 8 is added to each Second Strand cDNA Reaction from B3. cDNA Purification Spin Columns are inserted into Collection Tubes. The sample is pipetted onto each column and centrifuged for 1 min at maximum speed in a table top centrifuge. Note that guanidine thiocyanate in the Binding Buffer is an irritant. Always wear gloves and follow standard safety precautions to minimize contact when handling.

The flow-through is discarded and the columns are re-inserted in the same Collection Tubes. 500 μl Washing Buffer (with Ethanol added) is added to the columns and the columns are centrifuged for 1 min at maximum speed. The flow-through is discarded, the columns are re-inserted in the same Collection Tubes and washed with 200 μl Washing Buffer. The mixture is centrifuged for 1 min at maximum speed. The flow-through and the Collection Tubes are discarded.

The columns are inserted in fresh 1.5 ml reaction tubes and 50 μl of Elution Buffer is added to the columns. Elution Buffer is pipetted in the middle of the column, directly on top of the matrix, without disturbing the matrix with the pipette tip. The columns are incubated for 1 min, then centrifuged for 1 min at maximum speed. The columns are spun a second time with 50 μl Elution Buffer into the same reaction tubes, incubated 1 min and centrifuged again for 1 min at maximum speed. Eluate is transferred to fresh reaction tubes for further processing.

B5. Ethanol Precipitation of the Purified cDNA

The Precipitation Carrier (Tube 15) is stored in the dark. For long-term storage, the tubes are kept at −20° C. Smaller aliquots are kept at 4° C. for about 1 month. Precipitation Mix 9 is prepared according to the table below.

Precipitation Mix 9

| | | |
|---|---|---|
| Sodium Acetate | Tube 16 | 10 μl |
| Precipitation Carrier | Tube 15 | 2 μl |

12 μl of Mix 9 is added to each eluate (100 μl; from B4) and the mixture is mixed well. 220 μl of 100% ethanol (room temperature) is added, the mixture is mixed again, and incubated for 2 min at room temperature. The cDNA is centrifuged at maximum speed for 10 min at room temperature.

The supernatant is discarded and the pink-colored pellet is washed with 200 μl of 70% ethanol (room temperature). The tubes are centrifuged for 5 min at maximum speed and the supernatant is removed with a pipette.

To ensure that all liquid is removed, the tubes are spun briefly to collect liquid, and remaining liquid is removed with a pipette tip. The pellets are air dried by leaving the tubes open for about 5 min at room temperature. The pellets are not dried in a speed vacuum. The pellet is dissolved in 8 μl Solubilization Buffer (Tube 17) and kept at room temperature for further amplification. Alternatively the samples are stored at −20° C. for later use.

B6. Two Options for in Vitro Transcription Reactions

Two options are discussed to proceed with in vitro transcription reactions.

Reagents for 24× in vitro transcriptions with unmodified NTPs are included in the kit (first and second round, 12× each). Purification of amplified RNAs is performed with RNeasy Mini Kit (Qiagen®), as described by the manufacturer for “RNA Cleanup”.

Option 1) Affymetrix users apply the amplified cDNA (from step B5) as template for in vitro transcription with the ENZO Bioarray High Yield RNA Transcript Labelling Kit, according to the instructions of the manufacturer.

Option 2) Amplified labeled antisense RNA is obtained using the amplified DNA template (steps B4/B5) for in vitro transcription with an RNA labeling kit.

The extended Amino-Allyl Bacterial mRNA amplification kit (Cat.-No. 8092-A12) contains reagents to obtain amino-allyl-labeled, amplified RNA and to generate dye-coupled and fragmented RNA, ready for hybridization (this kit does not include the NHS-activated Dye-derivatives).

Troubleshooting

T1. RNA Isolation

To the extent possible, RNA is free of contaminating DNA. The Bacterial mRNA amplification kits are extremely sensitive to contaminating DNA fragments. A DNase treatment is combined with a spin column purification to remove fragments of digested DNA.

In general, satisfactory results may be obtained with the RNeasy Mini Kit from Qiagen (Qiagen Catalogue No. 74104) in combination with the RNase-Free DNase Set (Qiagen Catalogue No. 79254). Using this modified protocol, traces of DNA are directly removed on the spin column, followed by an additional wash step and final RNA elution.

In principle RNA isolated with Trizol (or RNA-Stat) protocols is essentially free of genomic DNA. However, this is not suitable for samples with degraded nucleic acids, because degraded DNA fragments will co-purify with RNA.

T2. RNA Quality with Large Samples

RNA isolation procedures should maintain the RNA quality in the samples. Whenever possible, the quality of purified RNA should be controlled by gel electrophoresis or with different technologies like the Agilent 2100 bioanalyzer (including the recently available RNA 6000 Pico LabChip). About 200-500 ng of total RNA is sufficient for agarose gel electrophoresis followed by ethidium bromide staining. For less RNA, more sensitive nucleic acid staining dyes or the Agilent 2100 bioanalyzer may be used.

For maintaining RNA quality during the isolation procedures, it is important to eliminate internal and external RNase activities. As soon as the cells are damaged, intracellular RNase activities will start RNA degradation. Immediately after sample collection, a lysis step should follow. To the extent possible, the samples are immediately shock-frozen with liquid nitrogen, followed by further storage at −80° C. The samples are not to be placed directly in a freezer after collection.

RNA degradation is minimized by as complete and rapid as possible sample lysis in strong denaturing agents like phenol, Trizol, RNAStat or guanidine thiocyanate (GTC). During microdissection, collected specimens are to be transferred immediately into a lysis reagent (supplemented with the N-Carrier of the ExpressArt® RNA CARE reagents).

External RNases are accidental contaminations. It is important to know that human finger tips are a rich source of external RNases. Thus, no equipment for RNA preparations is to be touched by hand without wearing gloves. Gloves should also be changed frequently.

Some guidelines for elimination of external RNases are discussed herein: To the extent possible, certified RNase-free reaction tubes as well as filtered pipette tips are to be used. Autoclaving reaction tubes and pipette tips is not recommended, due to potential risk of contamination with heat-resistant RNases. The RNA working area should be strictly separated from any other DNA work in a laboratory. Especially, performing plasmid preparations can contaminate the whole working area with the very stable, heat-resistant RNase A, because large amounts of this enzyme are routinely used in many protocols.

T3. RNA Quality Control with Very Small Samples including Microdissected Cells

The isolation of intact RNA from microdissected cells is generally more demanding than standard RNA preparations, due to the various steps of sample preparation, staining and microdissection. However, controlling the RNA quantity or quality may not always possible if only small cell numbers are collected (see section T2).

Furthermore, it might be almost impossible to predict RNA yields when working with microdissected cells. Yields may vary between 5% and up to 80% of the theoretical yield of about 0.1 picogram of total RNA per bacterial cell. The ExpressArt® PICO RNA CARE reagents are designed for optimal RNA yields and quality. Furthermore, with ExpressArt® mRNA Amplification kits, there should be less need for accurate quantitation of input total RNA.

For RNA quality control with tiny samples, two amplification rounds should be performed with the ExpressArt® Bacterial mRNA Amplification Kit. Subsequently, RNA quality control may be performed as described in step A9, and the examples shown above.

If there is no amplified RNA of satisfying quality, the yield or quality of the sample RNA preparation might not bee as high as expected. If possible, RNA preparation should be repeated with higher cell numbers.

T4. Problems with mRNA Amplification

No amplified RNA: With 50-100 ng input total RNA, the first amplification round yields enough material to detect an intense smear of amplified RNA in the gel with an aliquot (1-2 μl) of the transcription reaction (see bioanalyzer profiles). If no amplified material is observed, the kit reaction is performed again with the provided Positive Control RNA (Tube 23). If the control works properly, the sample RNA might have been RNase-contaminated. If the control also did not work, the protocol should be carefully followed. Starting with less than 50 ng total RNA, only the second round of amplification may yield visible amounts of amplified RNA.

Low yield of amplified RNA: Among different bacterial species, significant variations in the mRNA content may occur. Estimates range from 1% to 5% of total RNA, thus leading to different amplification yields even if the same amount of input total RNA is used. If only a faint, hardly visible, smear of amplified RNA in the gel is observed, but with the expected length distribution, a further amplification round may be considered, following steps B1-B5 of the protocol (this option is another advantage of our amplified RNA with defined sequences at each end).

Amplified RNA length too small: With the Bacterial mRNA Amplification kits, amplified RNAs should have a centre-of-mass between 0.2 and 1 kb.

Comparison of samples: Direct comparison of microarray data obtained from samples with different pre-treatments is avoided. Although relative changes in differential expression patterns are largely unaffected, samples without amplification or samples subjected to the same amplification procedures are compared directly. A unique advantage of ExpressArt® technology is the possibility to directly compare all amplified RNA samples, obtained with one, two or three amplification rounds.

Expected yields of amplified RNA include:

| Input total | aRNA | aRNA |
|---|---|---|
| RNA | 1st round | 2nd round |
| 200 ng | 4 ± 2 μg | with 500 ng aRNA 1st: 50 ± 20 μg |
| 100 ng | 2 ± 1 μg | with 500 ng aRNA 1st: 50 ± 20 μg |
| 50 ng | 1 ± 0.5 μg | with 500 ng aRNA 1st: 50 ± 20 μg |
| 10 ng | not detected | using all of aRNA 1st: 50 ± 20 μg |

Thermocycler profiles:

Before starting the ExpressArt® Bacterial mRNA amplification kit protocol, a thermocycler is programmed with the following temperatures and times. HOLD steps are included to provide time for thermal ramping or for adding reagents.

Thermocycler profile
for First Round Amplification

| Temperature | Time | Action |
|---|---|---|
| 65° C. | 4 min | Start of first cDNA synthesis |
| 37° C. | HOLD | add 10 μl First Strand cDNA Synthesis Mix 2 |
| 37° C. | 45 min | |
| 45° C. | 10 min | |
| 50° C. | 5 min | |
| 37° C. | 1 min | |
| 37° C. | HOLD | add 5 μl Primer Erase Mix 6 |
| 37° C. | 5 min | |
| 80° C. | 15 min | |
| 37° C. | 1 min | |
| 37° C. | HOLD | add 5 μl RNase Mix 3 |
| 37° C. | 20 min | |
| 37° C. | HOLD | add 15 μl Second Strand cDNA Synthesis Mix 4 |
| 96° C. | 1 min | |
| 37° C. | 1 min | |
| 37° C. | HOLD | add 5 μl Extender Enzyme A Mix 5 |
| 37° C. | 30 min | |
| 37° C. | HOLD | add 5 μl Primer Erase Mix 6 |
| 37° C. | 5 min | |
| 96° C. | 6 min | |
| 37° C. | 1 min | |
| 37° C. | HOLD | add 5 μl Primer C (Tube 12) |
| 96° C. | 1 min | |
| 37° C. | 1 min | |
| 37° C. | HOLD | add 5 μl Extender Enzyme B Mix 7 |
| 37° C. | 30 min | |
| 65° C. | 15 min | |
| 37° C. | 1 min | Spin to collect liquid |

End of cDNA-1 synthesis, continue with cDNA purification

Thermocycler profile
for Second Round Amplification

| Temperature | Time | Action |
|---|---|---|
| 65° C. | 4 min | Start of second cDNA synthesis |
| 45° C. | HOLD | add 10 μl First Strand cDNA Synthesis Mix2 |
| 45° C. | 30 min | |
| 70° C. | 15 min | |
| 70° C. | HOLD | place samples on ice |
| 37° C. | HOLD | add 5 μl RNase Mix 3, place samples in |
| | | thermocycler |
| 37° C. | 20 min | |
| 37° C. | HOLD | add 20 μl Second Strand cDNA Synthesis Mix 13 |
| 96° C. | 1 min | |
| 37° C. | 1 min | |
| 37° C. | HOLD | add μl Extender Enzyme B Mix 14 |
| 37° C. | 30 min | |
| 65° C. | 15 min | |
| 65° C. | HOLD | place samples on ice |

End of cDNA-2 synthesis, continue with cDNA purification

Thermocycler profile for optional Third Round Amplification is identical to Thermocycler profile for Second Round Amplification.

All patents, patent applications and references cited herein are incorporated by reference herein in their entirety.

We claim:

1. A method for the amplification of messenger ribonucleic acids (mRNAs), comprising:
(a) producing a first single-stranded DNA from a starting material comprising mRNA, using an RNA-dependent DNA polymerase, deoxyribonucleoside triphosphates, and a mixture of first single-stranded primers comprising the sequence 5′—a Box 1 sequence—1 to 6 random nucleotides—a specific trinucleotide sequence—3′;
(b) removing RNAs from the admixture of step (a);
(c) producing a first double-stranded DNA from said first single-stranded DNA using a DNA-dependent DNA polymerase, deoxyribonucleoside triphosphates, and a mixture of second single-stranded primers comprising the sequence 5′—a Box 2 sequence—1 to 6 random nucleotides—a specific trinucleotide sequence—3′, wherein said mixture of said second single-stranded primers differs from said mixture of said first single-stranded primers used in step (a);
(d) separating said first double-stranded DNA into second single-stranded DNAs;
(e) producing a second double-stranded DNA from one of said second single-stranded DNAs obtained in step (d), using a DNA-dependent DNA polymerase, deoxyribonucleoside triphosphates, and a third single-stranded primer comprising the sequence 5′—a promoter sequence—said Box 1 sequence—3′ or the sequence 5′—a promoter sequence—said Box 2 sequence—3′; and
(f) producing a plurality of first single-stranded RNAs, both ends of which comprise defined sequences of said Box 1 sequence or said Box 2 sequence, using an RNA polymerase and ribonucleoside triphosphates.

2. The method according to claim 1, wherein one or more of said plurality of first single-stranded RNA obtained in step (f) has an inverse sense orientation in relation to said mRNA in said starting material.

3. The method according to claim 1, wherein said Box 1 sequence is the same as said Box 2 sequence.

4. The method according to claim 1, wherein said Box 1 sequence is different from said Box 2 sequence.

5. The method according to claim 1, wherein said method yields a product mixture comprising ribonucleic acids and wherein said plurality of first single-stranded RNAs comprise more than 70% of the total amount of ribonucleic acids in said product mixture.

6. The method according to claim 1, wherein said method yields a product mixture comprising ribonucleic acids and wherein said plurality of first single-stranded RNAs comprise more than 80% of the total amount of ribonucleic acids in said product mixture.

7. The method according to claim 1, wherein said method yields a product mixture comprising ribonucleic acids and wherein said plurality of first single-stranded RNAs comprise more than 90% of the total amount of ribonucleic acids in said product mixture.

8. The method according to claim 1, wherein said RNAs are removed in step (b) using an RNase.

9. The method according to claim 1, wherein said ribonucleic acids are removed in step (b) using an RNase selected from the group consisting of RNase I and RNase H.

10. The method according to claim 1, wherein said Box 1 sequence or said Box 2 sequence contains at least 6 nucleotides and has a low homology to known gene sequences that are expressed in multi-cellular organisms.

11. The method according to claim 1, wherein said mRNA is selected from the group consisting of bacterial mRNA and eukaryotic mRNA.

12. The method according to claim 1, wherein said mRNA is a degraded mRNA.

13. The method according to claim 1, wherein said deoxyribonucleoside triphosphates are selected from the group consisting of dATP, dCTP, dGTP and dTTP.

14. The method according to claim 1, wherein said first double-stranded DNA in step (d) is separated into said second single-stranded DNAs using heat.

15. The method according to claim 1, wherein said third single-stranded primer in step (e) comprises a sequence of a T7 polymerase promoter sequence, a T3 polymerase promoter sequence, or a SP6 RNA polymerase promoter sequence.

16. The method according to claim 1, wherein said ribonucleoside triphosphates are selected from the group consisting of ATP, CTP, GTP and UTP.

17. The method according to claim 1, wherein the amplification factor of said mRNA is at least 500.

18. The method according to claim 1, wherein the amplification factor of said mRNA is at least 1000.

19. The method according to claim 1, further comprising:
(g) producing a third single-stranded DNA, using said first single-stranded RNAs produced in step (f), a fourth single-stranded primer comprising said Box 2 sequence, an RNA-dependant DNA polymerase and deoxyribonucleoside triphosphates;
(h) removing RNAs from the admixture of step (g);
(i) producing a third double-stranded DNA using said third single-stranded DNA produced in (g), a fifth single-stranded primer comprising the sequence 5′—a promoter sequence—said Box 1 sequence—3′, a DNA-dependent DNA polymerase and deoxyribonucleoside triphosphates; and
(j) producing a plurality of second single-stranded RNAs using an RNA polymerase and ribonucleoside triphosphates.

20. The method according to claim 19, wherein said RNAs in step (h) are removed using an RNase.

21. The method according to claim 19, wherein said second single-stranded RNA obtained in step (j) has an inverse sense orientation in relation to said mRNA in said starting material.

22. A method for nucleic acid analysis, comprising:
(a) obtaining ribonucleic acids;
(b) amplifying said ribonucleic acids using the method according to claim 1; and
(c) analyzing said amplification product obtained in step (b) using microarrays.

23. The method according to claim 22, wherein said ribonucleic acids are isolated from a biological sample.

24. The method according to claim 22, wherein the amount or sequence of said ribonucleic acids in step (a) is analyzed.

25. A method for nucleic acid analysis, comprising:
(a) obtaining ribonucleic acids;
(b) amplifying said ribonucleic acids using the method according to claim 19; and
(c) analyzing said amplification product obtained in step (b) using microarrays.

26. The method according to claim 25, wherein said ribonucleic acids are isolated from a biological sample.

27. The method according to claim 25, wherein the amount or sequence of said ribonucleic acids in step (a) is analyzed.

28. A method for nucleic acid analysis, comprising:
(a) obtaining ribonucleic acids;
(b) amplifying said ribonucleic acids using the method according to claim 1;
(c) converting said amplification product obtained in step (b) to cDNA; and
(d) analyzing said cDNAs using microarrays.

29. The method according to claim 28, wherein the amount or sequence of said ribonucleic acids in step (a) is analyzed.

30. A method for nucleic acid analysis, comprising:
(a) obtaining ribonucleic acids;
(b) amplifying said ribonucleic acids using the method according to claim 19;
(c) converting said amplification product obtained in step (b) to cDNA; and
(d) analyzing said cDNAs using microarrays.

31. The method according to claim 30, wherein the amount or sequence of said ribonucleic acids in step (a) is analyzed.
